(19) United States
(12) Patent Application Publication
(10) Pub. No.: US 2006/0004491 A1
(43) Pub. Date: Jan. 5, 2006

(54) Automated separation, purification and labeling system for 60Cu, 61Cu and 64Cu radionuclides and recovery thereof

(76) Inventors: Michael John Welch, Clayton, MO (US); Lucie Li Wa Tang, St. Louis, MO (US); Gregory Glen Gaehle, Chesterfield, MO (US); Jason Stuart Lewis, St. Louis, MO (US)

Correspondence Address: Patrick W. Rasche;Armstrong Teasdale LLP, Suite 2600, One Metropolitan Square, St. Louis, MO, 63102, US

(21) Appl. No.: 10/914,617

(22) Filed: Aug. 9, 2004

Related U.S. Application Data
(60) Provisional application No. 60/493,956, filed on Aug. 8, 2003.

(51) Int. Cl. G05B 21/00

(52) U.S. Cl. 700/266

(57) ABSTRACT

A novel method for separating an irradiated ⁶⁰Cu or ⁶¹Cu or ⁶⁴Cu respectively from a composition containing ⁶⁰Ni or ⁶¹Ni or ⁶⁴Ni respectively therein comprises dissolving the irradiated ⁶⁰Cu or ⁶¹Cu or ⁶⁴Cu in a solvent acid to form an acidic solubilized composition, feeding the acidic solubilized composition onto an ion exchange column and removing an eluent comprising ⁶⁰Ni or ⁶¹Ni or ⁶⁴Ni ions. In an aspect the eluent is further processed for ⁶⁰Ni or ⁶¹Ni or ⁶⁴Ni recovery and recycling to prepare future targets. In an aspect ⁶⁰Cu or ⁶¹Cu or ⁶⁴Cu respectively is temporarily trapped into the ion exchange column resin and held for subsequent recovery by addition of 0.5 N HCl to elute out the ⁶⁰Cu or ⁶¹Cu or ⁶⁴Cu for further use or labeling. An enhanced process for labeling compounds with highly purified ⁶⁰Cu, ⁶¹Cu or ⁶⁴Cu comprises loading ⁶⁰Cu, ⁶¹Cu and ⁶⁴Cu elute onto a concentrating cartridge, collecting the 0.5N HCl eluent and admixing therewith 10-μL of ligand and 3N HCl solution in a reaction line to form a ^{60 or 61 or 64}Cu labeled product. In an aspect a further purification step comprises loading 10-mL sterile water into the reaction line and through the C₁₈ Sep-Pak cartridge to further purify the labeled product which is adherent in the cartridge and recovering ⁶⁰Cu, ⁶¹Cu and ⁶⁴Cu each as a separate and independent purified product.

This application claims the benefit of U.S. provisional patent application 60/493,956 filed Aug. 8, 2003 which is incorporated herein in its entirety by reference.

### STATEMENT REGARDING FEDERALLY SPONSORED RESEARCH AND DEVELOPMENT

This work is supported by grants NIH/NCI R24 CA86307 and DOE DEFG02-87EF-60512. The government may have certain rights in the discovery.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

This discovery relates generally to a functional automated chromatographic process for automatically separating, purifying/refining and labeling copper radionuclides separately and independently and for isolating and recovering purified emitting copper radionuclides separately, independently and respectively as products.

The inventor relates to an enhanced process for recovery of enriched nickel radionuclides useful to produce (⁶⁰Cu, ⁶¹Cu and ⁶⁴Cu) radionuclides for recycling purposes. More particularly this discovery relates to an enhanced automated process for separating, purifying and recovering each of copper radionuclides ⁶⁰Cu, ⁶¹Cu and ⁶⁴Cu separately and independently respectively and for labeling compounds with purified ⁶⁰Cu, ⁶¹Cu and ⁶⁴Cu separately and independently respectively and to prepare a purified and labeled and recovered ⁶⁰Cu, ⁶¹Cu and ⁶⁴Cu separately and independently.

### BACKGROUND OF THE INVENTION

Each of radionuclides ⁶⁰Cu, ⁶¹Cu and ⁶⁴Cu respectively are utilized extensively in the treatment and diagnosis of cancer in living mammals. These radionuclides are useful for diagnosis (⁶⁰Cu, ⁶¹Cu and ⁶⁴Cu); internal radiation therapy (⁶¹Cu and ⁶⁴Cu) because of their positron—emission and/or toxicity to cancer and their characteristic intermediate half-life and multiple decay mode. Such therapies against cancer include the effective administration of radiolabeled chemicals requiring highly purified ⁶¹Cu and ⁶⁴Cu while therapies against cancer in mammals include the administration of ⁶¹ Cu and ⁶⁴Cu. ⁶⁴Cu (Copper-64) is especially useful in collaborative research and service projects and as a research tool as it can be distributed to multiple sites.

Despite advances over the years in developing processes in the chromatographic/liquid separation process field a strong need still remains for an automated separation, recovery, purification and labeling process for ⁶⁰Cu, ⁶¹Cu and ⁶⁴Cu. Additionally a need remains for an automated process which can effectively synthetically label ⁶⁰Cu, ⁶¹Cu and ⁶⁴Cu with appropriate ligands to make and recover therapeutic radiolabeled compounds for diagnosing or treating cancer or other diseases, such as stroke in a living mammal such as in a living human.

### BRIEF DESCRIPTION OF THE INVENTION

In an aspect the present discovery comprises a functional automated process for isolating and recovering ⁶⁰Cu, ⁶¹Cu and ⁶⁴Cu separately, independently and respectively each individually suitable for use in preparing radiodiagnostic agent(s) such for use in PET imaging and/or for use in preparing synthetic radiotherapeutic agents suitable for use in clinical applications involving use on living mammals. In an aspect the automated method is an automatic sequence system or a system emulating an automatic sequence system. In a further aspect the automatic sequence system is that control sequence shown in Table I. In an aspect ⁶⁰Cu, ⁶¹Cu and ⁶⁴Cu are isolated and recovered as products of this discovery. In an aspect, in this process each individual respective copper radionuclide is processed, separated, purified, isolated and recovered as its individual respective isolated ready to use (i.e., processed, purified, separated and recovered copper radionuclide).

In an aspect recovered purified ⁶⁴Cu having a purity level graded in specific activity in mCi/μg is produced in the grade range from about 20 mCi/μg to about 200 mCi/μg of Cu and preferably near 200 mCi/μg of Cu.

In an aspect an automated functional method for enhanced separating of an individual radioactive ⁶⁰Cu containing ⁶⁰Ni respectively, or an individual radioactive ⁶¹Cu containing ⁶¹Ni respectively, or an individual radioactive ⁶⁴Cu containing ⁶⁴Ni therein respectively comprises dissolving such individual irradiated ⁶⁰Cu containing ⁶⁰Ni, or such individual ⁶¹Cu containing ⁶¹Ni, or such individual ⁶⁴Cu containing ⁶⁴Ni mixture in a solvent acid to form an acidic solubilized composition, feeding/loading the respective acidic solubilized composition onto an ion exchange column and removing an eluent comprising individual ⁶⁰Ni, or individual ⁶¹Ni, or individual ⁶⁴Ni ions respectively and recovering the individual ⁶⁰Cu, individual ⁶¹Cu and individual ⁶⁴Cu respectively to provide invidivually and separately recovered ⁶⁰Cu, ⁶¹Cu and ⁶⁴Cu each individual respectively an independently, isolated and purified product from its original Ni containing respective starting material.

An automated functional separation system comprising a chromatographic separation zone further comprising a resin having a sufficient distinctive binding capacity for a ⁶⁰Cu, or ⁶¹Cu, or ⁶⁴Cu over ⁶⁰Ni, or ⁶¹Ni, or ⁶⁴Ni respectively and having a separation capability effective to substantially chromatographically high efficiency separate precursor ⁶⁰Ni, or ⁶¹Ni, or ⁶⁴Ni from ⁶⁰Cu, or ⁶¹Cu, or ⁶⁴Cu respectively to prepare ⁶⁰Cu, or ⁶¹Cu, or ⁶⁴Cu. In an aspect the automated method is an automatic sequence system. In a further aspect the automatic sequence system is that control sequence shown in Table I.

The discovery is additionally directed to a functional automated process for synthetically forming a ⁶⁰Cu, or ⁶¹Cu, or ⁶⁴Cu labeled product which comprises loading purified ⁶⁰Cu, or ⁶¹Cu, or ⁶⁴Cu in 0.5N HCl solution onto a concentrating assembly, removing an about 0.5N HCl eluent, adding 3N HCl thereto, and admixing about 10-μL of ligand solution with the highly purified ⁶⁰Cu, or ⁶¹Cu, or ⁶⁴Cu in the concentrating assembly forming a reaction system. The mixture formed with ⁶⁰Cu, or ⁶¹Cu, or ⁶⁴Cu in about 3N HCl/ligand is loaded onto a purifying cartridge removing an about 3N HCl eluent. In an aspect a further purification step comprises loading 10-mL sterile water into the reaction assembly. To remove the ⁶⁰Cu, or ⁶¹Cu, or ⁶⁴Cu labeled product which is adherent in the reaction assembly, ethanol is loaded onto the purifying cartridge. In an aspect the assembly comprises concentrating and purifying cartridges. In an aspect the system comprises a line or reaction chamber comprising a lumen for suitably reacting products therein.

In an aspect a method of controlling a functional automated process for synthetically forming a ⁶⁰Cu, or ⁶¹Cu, or ⁶⁴Cu labeled product comprises loading purified ⁶⁰Cu, or ⁶¹Cu, or ⁶⁴Cu in 0.5N HCl solution onto a concentrating assembly, removing an about 0.5N HCl eluent, adding 3N HCl thereto, and admixing about 10-μL of ligand solution with the highly purified ⁶⁰Cu, or ⁶¹Cu, or ⁶⁴Cu in the concentrating assembly forming a reaction system which comprises forming a database containing sequence control information and using that database to control the process. In an aspect the process comprises a process for operating a chromatographic column. In a further aspect the column is a separation column for copper nuclides.

In an aspect a database comprises a functional sequence valve instruction useful for controlling an automated process for synthetically forming a ⁶⁰Cu, or ⁶¹Cu, or ⁶⁴Cu labeled product which comprises loading purified ⁶⁰Cu, or ⁶¹Cu, or ⁶⁴Cu in 0.5N HCl solution onto a concentrating assembly, removing an about 0.5N HCl eluent, adding 3N HCl thereto, and admixing about 10-μL of ligand composition with the highly purified ⁶⁰Cu, or ⁶¹Cu, or ⁶⁴Cu in the concentrating assembly forming a reaction system. In an aspect the database comprises a sequence of functional coordinated valve openings and valve closings. In a further aspect the automatic sequence system is that control sequence shown in Table II or a system emulating that system.

### BRIEF DESCRIPTION OF THE DRAWINGS

FIGS. 1 and 2 illustratively comprise schematic block diagrams of the automated processes of this discovery.

FIG. 1 is a schematic which depicts an automated operative copper radionuclide separation and purification unit schematic having components capably coupled together and useful for separating ⁶⁰Cu from unreacted ⁶⁰Ni target material or, for separating ⁶¹Cu from unreacted ⁶¹Ni target material or, for separating ⁶⁴Cu from unreacted ⁶⁴Ni target material from other radionuclides.

FIG. 2 is a schematic having components capably operatively coupled together which depicts an automated operative copper radionuclide labeling unit schematic.

FIG. 3 is a Table I showing an automated copper separation and purification sequence useful in this discovery.

FIG. 4 is a Table II showing an automated operative copper labeling sequence useful in this discovery.

FIGS. 5A, 5B, 5C and 5D are dimensioned schematics of different views of a syringe holder designed and utilized by the inventors.

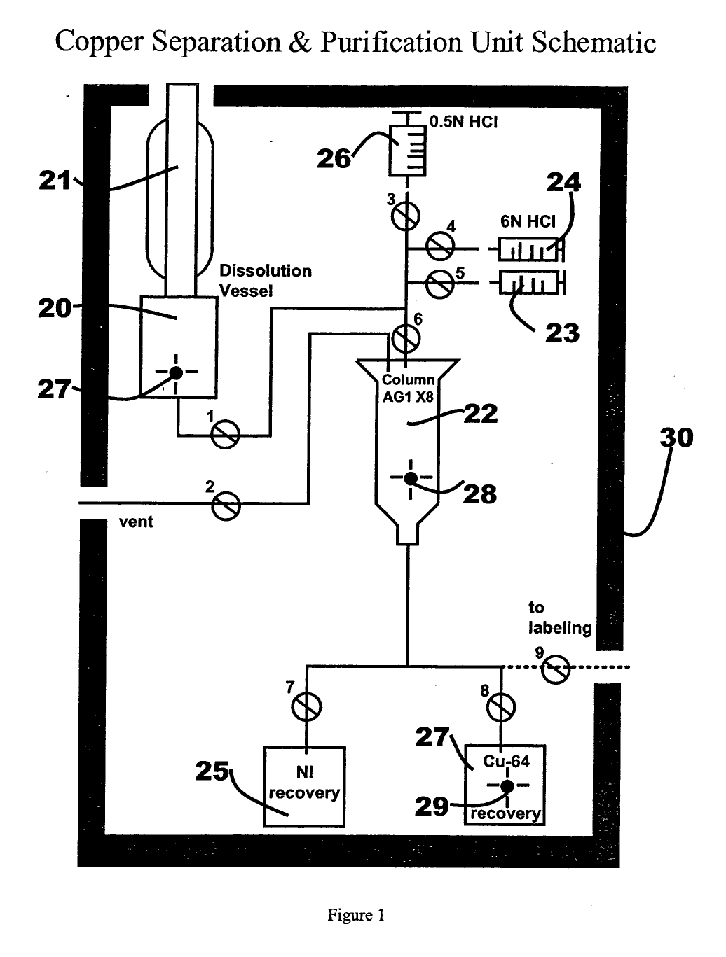

The discovery is described hereinafter in further detail with references to the aforedescribed FIGS. 1-5A, 5B, 5C and 5D in which like items are number the same in the aforedescribed FIGS. 1-5A, 5B, 5C and 5D.

### DETAILED DESCRIPTION OF THE INVENTION

In an aspect radioactive ⁶⁰Cu, ⁶¹Cu and ⁶⁴Cu are isolated and recovered as purified products of this discovery for further use such in a radiolabel tracer compound.

In an aspect this discovery comprises two stand alone unit(s) in an automated system which can be operated together. In an aspect a first stand alone unit is a functional automated copper radionuclide separation and purification process. In an aspect a second stand alone unit is a functional automated copper radionuclide labeling process. In an aspect radioactive ⁶⁰Cu, ⁶¹Cu and ⁶⁴Cu are isolated and recovered as purified products of this discovery for further use such in a radiolabel tracer compound. In an aspect the automated system is electrically/pneumatically communicatively configured capable and functional in all operationally necessary aspects.

More in detail each of the respective copper radionuclides (60, 61 and 64) are produced from a different but respective enriched Ni target material for example: ⁶⁰Cu is produced from ⁶⁰Ni via the nuclear reaction ⁶⁰Ni(p,n)⁶⁰Cu; ⁶¹Cu is produced from ⁶¹Ni via the nuclear reaction ⁶¹Ni(p,n)⁶¹Cu and ⁶⁴Cu is produced from ⁶⁴Ni via the nuclear reaction ⁶⁴Ni(p,n)⁶⁴Cu. In an aspect ⁶¹Cu is also produced by the ⁶²Ni[d,n]⁶¹Cu nuclear reaction.

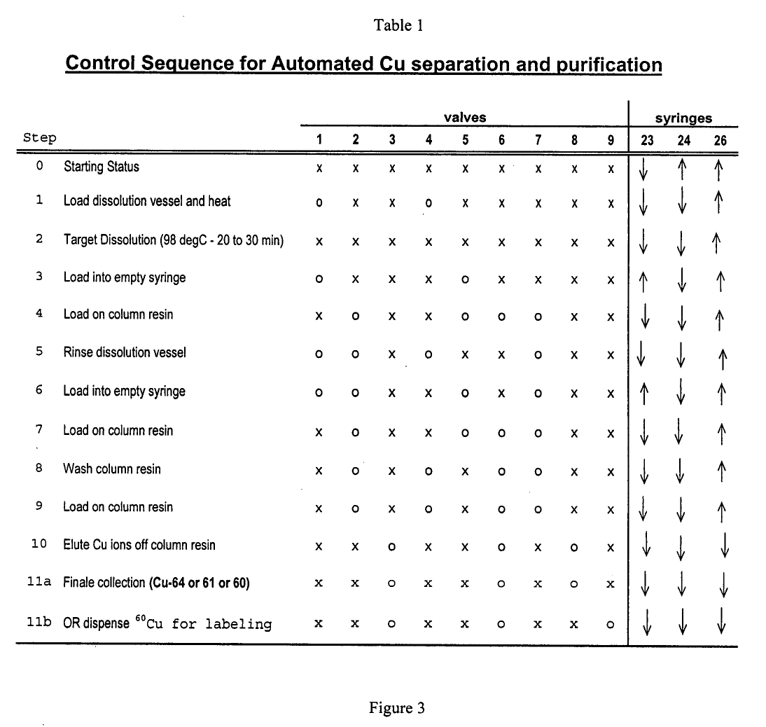

In an aspect the first unit is programmed to operate a process scheme utilizing process equipment and an arrangement of the copper radionuclide separation and purification unit schematic of FIG. 1 using the automated copper separation and purification sequence shown in Table I (FIG. 3).

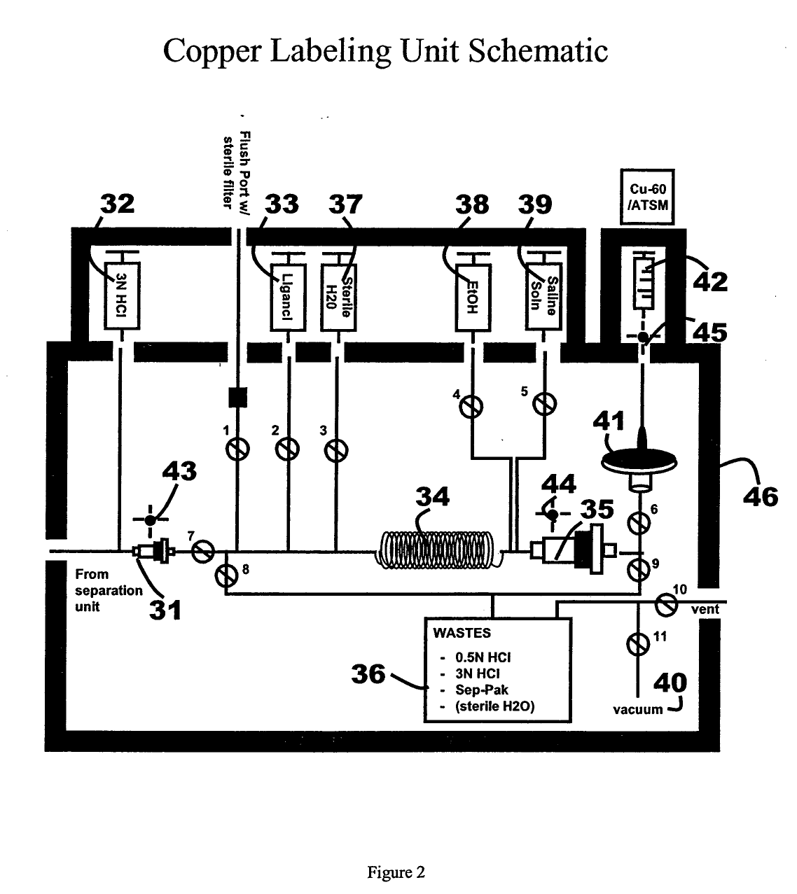

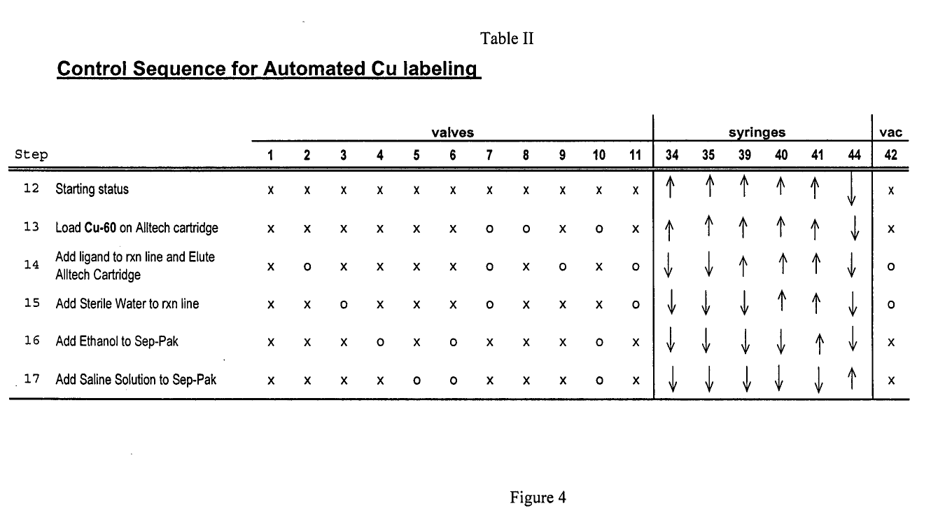

In an aspect a second unit is programmed to capably operate utilizing process equipment and an arrangement of the copper radionuclide labeling unit schematic shown in FIG. 2 using the automated copper radionuclide labeling unit schematic shown in Table II (FIG. 4).

It is understood that automatic control systems are employed which have been operably loaded with effective functional software, coupled through functional electrical mechanical servo-mechanisms directed by the software commands to appropriate valves on the respective units to carry out an automated operation of the unit. It is further understood that an operating functional computer may be suitable employed in this automatic control system having sufficient memory to carry out the software commands being suitably connected to the valves and solenoids through a cooperative and capable operating effectively communicating network using the schematics and sequences such as described herein providing the needed process flows and no flows.

In an aspect a functional computer includes one which has the capability to house and operate the software necessary for this discovery and is fully communicative with all valves and associated tubing and equipment. It is understood that the term communicative includes full capability of sending, receiving and providing timely instruction to/from the equipment in accordance with the software and this discovery.

In an aspect the operation is equipped with valves which are pneumatic actuated pinch valves. Once energized, the piston will pinch the tubing located in its holder which corresponds to a closed status. In an aspect the aforedescribed database, software, valves, vessels, nuclides and starting materials are assembled and setup in an operating assembly.

In an aspect there is herein described the capability functions of the software for operating the automated process. The functions include those for operating the computer, (such as a PLC), the software and for communication but also those illustrated in the Examples and in the specification, claims and drawings including those schematics presented in Tables I and II attached including valve sequences for operations including timing of valve positions, sequencing of valves, opening and closing at appropriate time of valves, cycling of valves and the like. The functions of the software herein presented provide those of skill in the art after reading the specification and claims the ability to make and use this discovery.

Any commercial PLCs with their respective software programming language can be used as a computer to on which program the control sequence for this application and performs the automated process control of this discovery. Useful commercial PLC's include those from Allen-Bradley/Rockwell Automation (1201 So. Second, St. Milwaukee, Wis. 53204-2496 USA), Omron Electronics LLC (Schaumburg, Ill. USA), Crouzet (Coppell, Tex. USA), Automation Direct (Cumming, Ga. USA), Mitsubishi Electric Automation Inc. (Vernon Hills, Ill. USA), Motorola Inc. (Tempe, Ariz. USA) and Siemens Motion Control Systems (Elk Grove Village, Ill. USA), etc. In an aspect these are equipped with a chip and useful PLC programming software.

For example useful PLC programming software includes IEC 1131-3, Sequential Function Chart (SFC), Function Block Diagram (FBD), Ladder Diagram (LD), Structured Text (ST), Instruction List (IL), Relay Ladder Logic (RLL), Flow Chart and Basic. These softwares are usable on the above noted PLC's.

In as aspect PLC (Programmable Logic Controller) is a device designed to perform logic functions which performs as an instruction unit. PLCs are then digital electronic control systems for a wide variety of automated systems and processes and are equipped with multiple input and output interfaces and a control programming. In an aspect, the sequences of this discovery are programmed into the PLC software program language for implementation and automatic operator/control of this novel system comprising a PLC.

In an aspect a PLC from Allen-Bradley (catalog # 1747-L541, Milwaukee, Wis.) is employed having its software RSLogix 500 (catalog # 9324RL0300ENE) from Rockwell Automation (Milwaukee, Wis.) programmed with the sequences of this discovery to carry out automation of both units of this discovery.

Example of Valve Sequence and Temperature Control for Step 1 (Load Dissolution Vessel and Heat) (See Table I):

Energize valves 2, 3 and 5 to 9 to pinch tubing and close the pathway. De-energize valves 1 and 4 to open the pathway for 6N HCl acid in prefilled syringe 24 (plunger in pushing status) to flow into the dissolution vessel. Once the required volume of 6N HCl acid is reached, energize valves 1 and 4 to close the pathway. All valves are in a closed status. Energizing valves causes the valves to close and thus pinch tubing and close the pathway. Start the heating of the acid until it reaches ˜100° C. Using a three-mode control action PID (Proportional, Integral, Derivative) programmed into the PLC keep the temperature around this set value until ready to go to next step, dissolution of the irradiated target.

Example of Valve Sequence Step 11b (Dispense Cu-60 for Labeling) (see Table I):

Energize valve 8 to close the pathway into recovery vessel and de-energize valve 9 to open the pathway for purified copper radionuclide in 0.5N HCl to flow into the second automated unit—automated labeling. Prefilled syringes 23, 24 and 26 are in pushing plunger status. Syringes 23 and 24 are in final status from previous steps and syringe 26 prefilled with 0.5N HCl is eluting the column.

Example of Valve Sequence Step 1 (Load Cu-60 onto Alltech Cartridge) (See Table II):

Prior to receiving the purified copper radionuclide in 0.5N HCl from the first automated unit (separation and purification unit), ready the labeling unit by executing the following sequence: energize valves 1 to 6, valve 9 and valve 11 to pinch tubing and close the pathway. De-energize valves 7,8 and 10 to open the pathway for purified copper radionuclide to flow through the concentrating cartridge (Alltech). The purified copper radionuclide is retained onto the cartridge while the 0.5N HCl flows into the vented wastes container. Pre-filled syringes 32, 33, 37, 38 and 39 are in pulling plunger status (no dispensing of reagents) and empty syringe 42 is in pushing plunger status (no filling). The vac (vacuum) is in close status indicating no vacuum is used during step.

PID Control is a three-mode (Proportional-Integral-Derivative) control action which tunes automatically a variable to hold the measurement at a setpoint value which is where the measurement is assigned to be, for example the temperature in this application. Proportional control continuously adjusts the output dependent on the relative measurements of the process and the setpoint.

As used herein the term “PID control” includes an algorithm used for the control process loops in the process of this discovery. PID is useful for this basis advanced control algorithm. One would tune the PID algorithm to this discovery.

As used herein, the term “PID” means respectively proportional, integral and derivative. The starting point is a setpoint value which is the point, place or status where the human operator would like the controlled variable, or process to be. We determine error which is the difference between desired setpoint and measurement. This can be expressed mathematically as (error)=(setpoint)−(measurement). The variable being adjusted is called the manipulated variable which usually is equal to the output of the controller.

The output of functioning PID controllers will change in response to a change in measurement or setpoint according to modes of the controller. Modes are denoted as: P, proportional band which is referred sometimes as gain which is the reciprocal of proportional band and is defined as 100/gain (% units), I, integral which is a function which adjusts the controlled variable to setpoint after the system stabilizes and can be defined as one/reset and D, derivative which senses the rise or fall of the system variable and automatically adjusts the P to minimize variation and is also known as rate which is also pre-act (units are of time). Integral and reset are the same and are in time/repeat or repeat/time with integral being the reciprocal of reset and vice versa. Derivative and rate are the same.

Illustrative useful process and temperature control product lines include those of West Instrument, LFE, Watlow and Gentran, USA.

Any useful capable existing temperature control techniques such as on/off, P (proportional) or PD (Proportional-Derivative) can be employed.

In an aspect a first automated unit as its automation control system using a computer such as a PLC having its PLC software programmed in accordance with this invention to capably carry out the automation described herein, collects the dissolved activity off the irradiated target into a target solution. The target solution is loaded onto an ion exchange column for separation and purification through sequential use of selective reagents (mobile phase carriers) The activity is collected in a collection container or vial for further research function or, is directed into a second automated unit.

In an aspect a novel second automated unit (different from or the same as a first) such as a computer such as a PLC having its PLC software programmed in accordance with this invention performs the labeling of the recovered radioactivity with an appropriate ligand or biomolecule. The ligand as such is designed that complexation between the copper nuclide (⁶⁰Cu, or ⁶¹Cu, or ⁶⁴Cu) and the ligand or biomolecule produces a copper-labeled ligand or biomolecule (compound) that will have pharmacological distribution suitable for the effective diagnosis or treatment of disease. In that regard an illustrative example of the novel labeling method of this discovery is an automated labeling (producing) a copper complex such as Copper (II)-diacetyl-bis(N-methylthiosemicarbazone) (aka Cu(II)ATSM), which is a promising agent for imaging hypoxic mammalian tissue. See J. Nucl Med 1999 40:177-183. Subsequent use of different cartridges, filters and reagent gives a final purified sterile and pyrogen free product ready for injection to a patient in the case of ⁶⁰Cu-ATSM.

In an aspect the pharmaceutical composition comprises a (purified if desired) tracer compound optimally with an emitting radiolabel such as a copper nuclide and optionally a suitable adjuvant such as a surfactant which is pharmacologically acceptable to the patient such as to a living mammal such as a living human.

In an aspect, first automated and second automated units are operated as automated stand alone units or optionally coupled and operated automated in an effective two unit arrangement. An automatic system may be employed to operate each of the units as a stand alone or optionally as a coupled unitized and integrated automatic unit.

In an aspect the first unit is automated. In an aspect the second unit is automated. In an aspect both first and second units are automated. In an aspect only one unit is automated.

In an aspect the first automated and second automated units are operated as an automated unit comprising a combined automated system automatic control systems which have been operably loaded with effective software, coupled through electrical mechanical servo-mechanisms directed by the software commands to appropriate valves on the respective units to carry out an automated operation of the unit. It is further understood that a computer may be suitably employed in this automatic control system having sufficient memory to carry out the software commands being suitable connected to the valves and solenoids through a cooperative and capable operating network.

As used herein, the term “purified ⁶⁴Cu” has a—specific activity—in the range from about 20 mCi/μg to about 200 mCi/μg and preferably at least about 20 mCi/μg for use as an imaging agent and a higher specific activity ranging from about 150 mCi/μg to about 200 mCi/μg for use as a therapeutic agent.

As used herein the term “chromatography” includes techniques involving mass-transfer between one or more stationary phases and one or more mobile phases such as typically carried out in a chromatographic separation zone. As used herein, the term “chromatography” includes any useful form that uses a column or tube or container having an internal lumen to satisfactorily hold a stationary phase. Useful illustrative chromatographic techniques include open column chromatography, HPLC and open tubular capillary chromatography.

In an aspect the column comprises a borosilicate (glass) Econo-column from Bio-Rad having catalog number 737-1031. Other sizes and material construction of columns can be employed for this application. In more detail the 737-1031 chromatographic column is 1.0×30 cm, 24 ml. About 4 cm of packing material is used in the column or 2.74 to 2.76 grams and preferably near 2.75 grams of packing material. Packing support which is understood to be a porous polymer bed support is manually packed in the column. In an aspect the column has translucent polypropylene end fittings (such as Luer-Lok) which allow visualization of the column bed. Another illustrative useful column is a jacketed Econo-Column which is another type of Econo-Column from Bio-Rad and which has an integral water jacket.

The term “chromatographic separation zone” is employed herein to mean any zone capable of effecting a separation of the components of a multi-component composition and includes useful chromatographic zones such as chromatographic columns of any useful shape, size, description or composition.

As used herein, the term “column” includes a plastic or glass high normality hydrochloric acid resistant tube or rounded container having a lumen therein with polished inner surface and fittings at both ends suitably configured for packing with small porous adsorbent particles as column packing therein.

The term “packing” is employed throughout this application and includes any ion exchange resin or any suitable retaining material employed in the internal volume of a chromatographic separation zone which is capable of retaining thereon a component of interest (copper radionuclide) releasable from the packing upon elution with an appropriately selected mobile phase carrier.

The term “multi-component composition” is employed throughout to mean a composition containing more than one component and includes compositions such as mixtures as well as true solutions.

As used herein, the term “preparation, synthesis, purification and recovery” to such a state/condition ready for use such as use as a radionuclide with a tracer compound for diagnostic imaging in animals.

In an aspect packing employed in a chromatographic separation zone in a first aspect of this discovery has a particle size diameter in the range from about 30 to about 1000 microns and preferably from about 35 to about 400 microns.

The type of packing as retention support material which may be employed in the chromatographic separation zone and any second chromatographic separation zone is selected to retain a component of interest within a discreet zone of the packing which is releasable upon sequential elution with an appropriately selected mobile phase carrier after reading this specification. In an aspect the packing is selected to temporarily retain Copper-60, or Copper-61, or Copper-64 which is sequentially and selectively releasable from such temporary retention by passing an appropriate mobile phase carrier over the packing containing the Copper-60, or Copper-61, or Copper-64. Typical useful nonlimiting packing includes polystyrene, divinyl benzene resin and silica base packing.

In an aspect, packing employed comprises Bio-Rad AG® 1-X8 Resin, 100-200 mesh chloride from catalog 140-1441, Bio-Rad Laboratories, 2000 Alfred Nobel Drive, Hercules, Calif. 84547. The resin is a styrene type—quaternary ammonium having a medium effective pore size with a Total Capacity of 2.6 meq/dry g, 1.2 meq/ml resin bed, Actual Wet Mesh Range of 80-140 (US Std) 106-180 microns, Moisture content of 39-48% by wt. and density (nominal) 0.75 gm/ml.

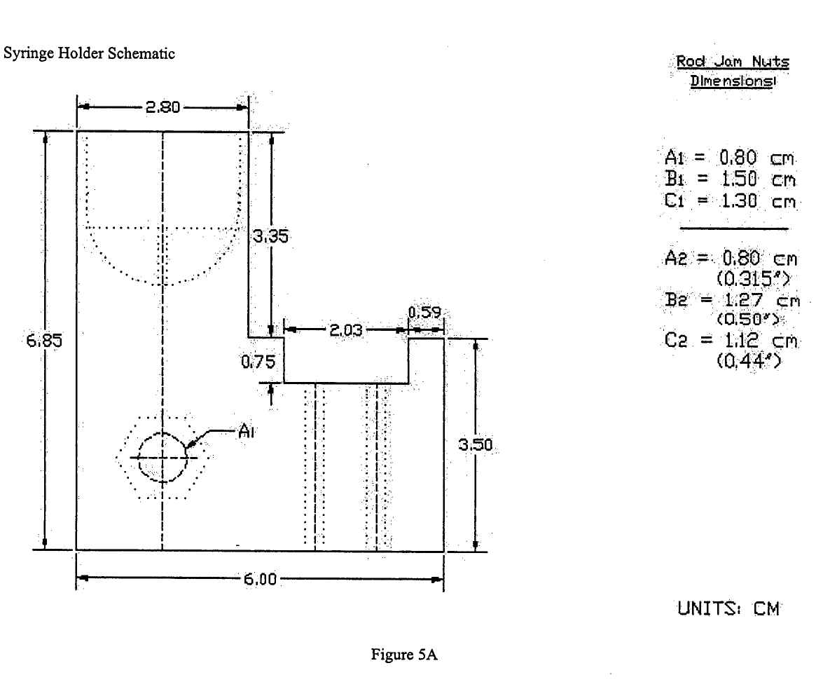

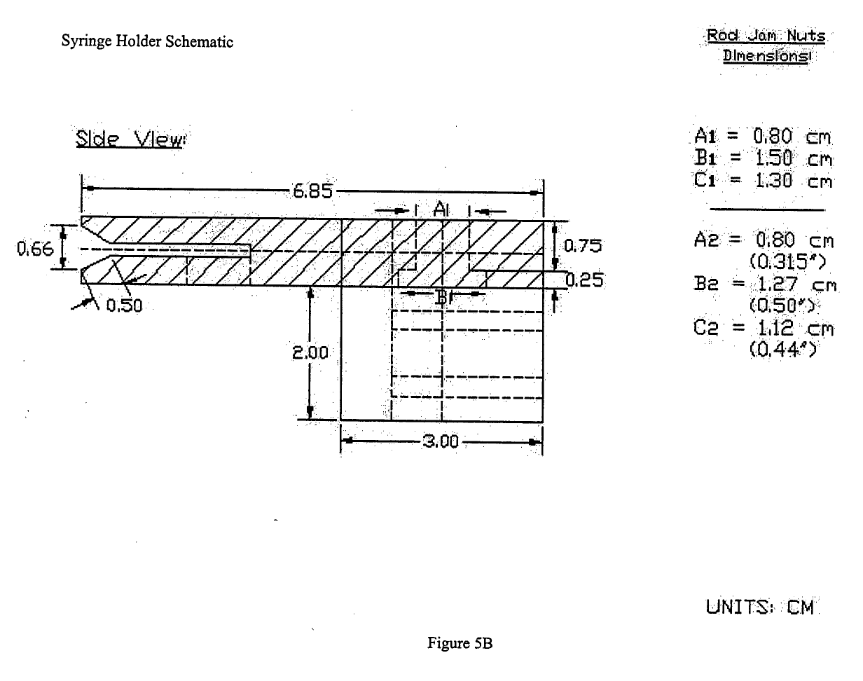

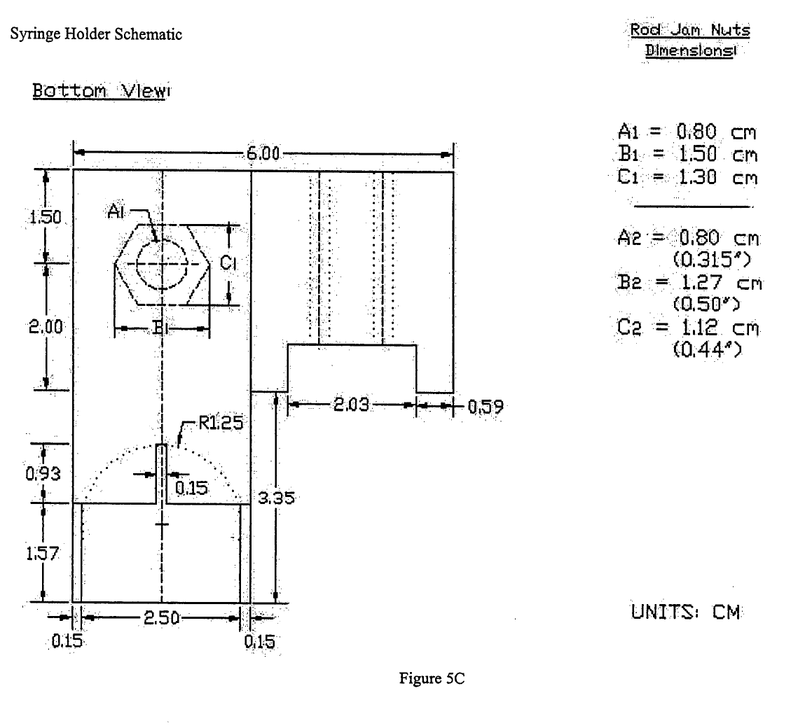

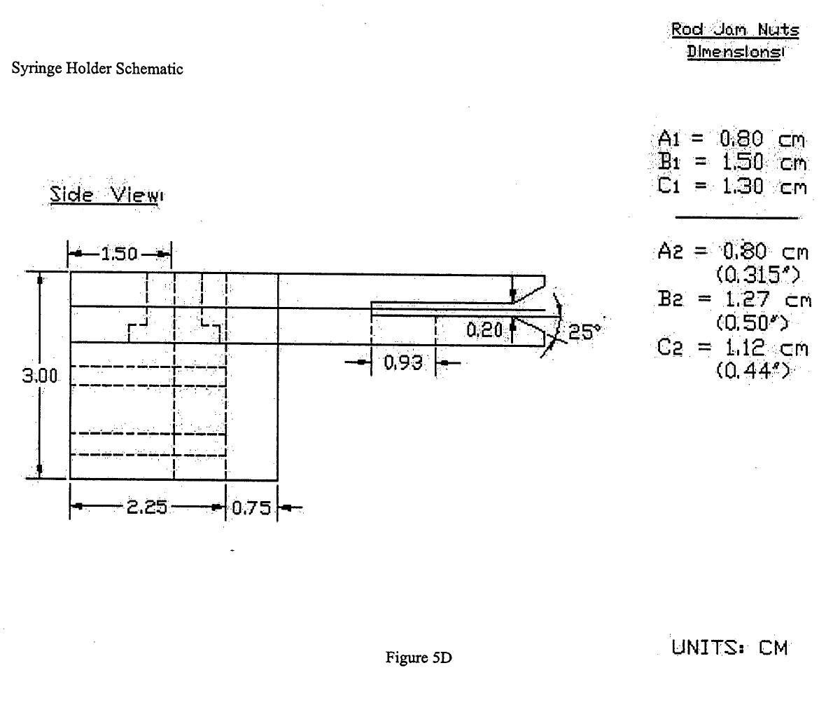

In an aspect, pneumatic parts such as pinch valves and air cyclinders were supplied by SMC Pneumatics, Inc., 3011 N Franklin Road, Indianapolis, Ind. 46226. PLC, analog and digital modules, 110 volt power supply, chassis were supplied by Allen-Bradley Company, LLC, Rockwell Automation, 1201 South Second street, Milwaukee, Wis. 53204. The vacuum pump was supplied by Vaccon Co., Inc. 32 Rear Spring St, Medfield, Mass. 02052. Syringes Norm-Ject® were supplied by Air Tite Products Co, Inc, 565 Central Drive, Virginia Beach, Va. 23454. The syringe holder (FIG. 5) was machined at Washington University in St. Louis, One Brookings Drive, St. Louis, Mo. 63130 from material resistant to acid. A schematic for the syringe is presented in FIG. 5.

H₂-ATSM was produced at Washington University in St. Louis by a method following the literature procedure of Gingras B A, Suprunchuk T, Bayley C H. Can J. Chem Part III 40, 1053-1059 which is incorporated herein in its entirety by reference.

In relation to ATSM noted above, in an aspect, the procedure employed in the synthesis of H₂ATSM is based on a method described in the literature by H. G. Petering et al., B. A. Gingas et al., and F. A. French, et al. In brief, 4-Methyl-3-thiosemicarbazide is dissolved in 50-mL 5% acetic acid and maintained at a temperature of 50-60° C. with constant stirring. 2,3-Butanedione is taken up in 10-mL MilliQ water and added dropwise to the 4-methyl-3-thiosemicarbazide solution over a 45 minute period. Soon after the butanedione addition is started, a precipitate begins to form in the light yellow solution. The mixture is left stirring for an additional 30 minutes at 60° C., and then the hot solution is filtered through a coarse fitted-glass filter to isolate the solid product. The isolated H₂ATSM is washed with 2×50-mL water and then 2×50 mL ethanol and dried at 75° C. H₂ATSM is recrystallized by taking up into 100-mL 80% acetic acid and heated under reflux for 30 minutes. The solution is filtered hot and any undissolved material is collected and dried at 75° C.

The term “mobile phase carrier” is employed throughout this application to include any composition capable of being passed into a chromatographic separation zone to effect the elution of a compound temporarily retained in the packing of a chromatographic separation zone. Typically the mobile phase carrier is a liquid or in liquid form at the time of being passed.

In an aspect the particular mobile phase carrier associated with first chromatographic separation zone corresponds with the type of packing employed in a first chromatographic separation zone.

As used herein, the term “detectably labeled” includes respective highly purified ⁶⁰Cu, ⁶¹Cu and ⁶⁴Cu labeled compounds having an effective amount of an emitting copper radionuclide radiolabel therewith suitably accommodating for use in effective administration/therapy to living mammals. In an aspect, small animal imaging using copper radionuclides (⁶⁰Cu, ⁶¹Cu and ⁶⁴Cu) is done on rodents (including mice and rats) following administration thereto of copper radiopharmaceuticals.

As used herein, the term “small animal” imaging includes imaging done on cats, dogs, mice, rats and rodents. As used herein the term “rodent” includes members of the Order of Rodentia including squirrels, rats, prairie dogs, porcupines, mice, lemmings, marmots, guinea pigs, hamsters, gophers, gerbils, chipmunks, chinchillas, beaver, capybaras, porcupines, ground squirrels and beaver.

As used herein, the term “administration” includes the successful giving of an individually highly purified ⁶⁰Cu, ⁶¹Cu or ⁶⁴Cu labeled compound by any useful means to a living mammal and its successful introduction into the mammal internally such as by intravenous injection in an effective method which results in that compound, its salt, its ions, metabolites or derivatives being made biologically available to that mammal receiving administration of the highly purified ⁶⁰Cu, ⁶¹Cu and ⁶⁴Cu labeled compound for medicinal or therapeutic use. In an aspect the mammal is a living nonhuman mammal such as a canine, feline, rat, rodent, mouse or a living cell therefrom. In an aspect the highly purified ⁶⁰Cu, ⁶¹Cu or ⁶⁴Cu labeled compound is made biologically available to the mammal patient. In an aspect the administration comprises giving of at least one of a highly purified ⁶⁰Cu, ⁶¹Cu or ⁶⁴Cu detectably labeled compound. In an aspect, the mammal is a human and the radionuclide is individually Copper-60 or Copper-61 or Copper-64.

As used herein, the expression “pharmaceutically acceptable” applies to a composition comprising a compound or its copper radiolabeled counterpart herein which contains composition ingredients that are compatible with other ingredients of the composition as well as physiologically acceptable to the recipient, e.g. a mammal such as a human. In an aspect, a composition for use comprises one or more carriers, useful excipients and/or diluents. In an aspect the composition comprises at least one of a ^{60, 61 or 64}Cu detectably labeled compound.

In an aspect the pharmaceutical composition comprises a purified tracer compound with an emitting radiolabel and optionally a suitable adjuvant such as a surfactant which is pharmacologically acceptable to the patient such as to a living mammal such as a human. The pharmaceutical may comprises a water soluble salt of a tracer compound in an aqueous with an associated emitting radiolabel as well as a saline solution. High purity radiolabel and high activity radiolabel are preferred. The choice of tracer compound and radiolabel will be determined to an extent by the particular affliction being diagnosed, such as cancer.

As used herein, the term “dosage” includes that amount of automatically separated, recovered and purified ⁶⁰Cu, ⁶¹Cu and ⁶⁴Cu compound which when effectively administered to a living mammal provides an effective amount of biologically available ⁶⁰Cu, ⁶¹Cu and ⁶⁴Cu labeled compound to the living mammal to enable radioimage detection and acquisition via external radioimage detector.

In an aspect, as used herein the term “patient” includes a human and a non-human such as feline, canine, horse and murine.

In an aspect as used herein the term “tissue” includes mammalian body tissue of the mammal being administered the radiolabeled compound.

In an aspect this discovery provides an enhanced process for automatically separating, purifying and recovering radionuclides from a multi-component composition and labeling individual radionuclides. In an aspect the discovery method utilizes liquid chromatography to selectively separate Nickel-60 from a mixture of Copper-60 and Nickel-60, or to selectively separate Nickel-61 from a mixture of Copper-61 and Nickel-61, or to selectively separate Nickel-64 from a mixture of Copper-64 and Nickel-64. Copper-64 is useful in clinical, major medical treatment and/or research facilities as it can be distributed to multiple sites and as a radionuclide for pharmaceutical.

In an aspect Liquid Chromatography (LC) herein is utilized as a mode of chromatography on a multi component feed composition containing a precursor nuclide of nickel and a nickel bombardment product being a radionuclide of copper.

In separating radionuclides LC utilizes a liquid mobile phase to successfully effectively separate the components of a mixture such as a mixture of Copper-64 and Nickel-64. The Nickel-64 and Copper-64 components (or analytes) (or Nickel-60 in Copper-60 or Nickel 61 in Copper-61) are dissolved in a solvent, and fed to a chromatrographic column under atmospheric pressure or gravity. In the column, the mixture is resolved into its components. In an aspect the stationary phase is immobile packing material in the column. In an aspect, the immobile packing material is held in place by an appropriate packing support in the lumen of the column. In an aspect the immobile packing material is purchased as a part of the column or added to the lumen of the chromatographic column prior to loading of the components to be separated. The pressure in the column is generally atmospheric pressure in the range from about 1 to about 2 atmospheres (14.7 to 29.4 psi respectively). In an aspect, the column is a vented column and elution is by gravity. Also the column can be pressurized up to about 2.5 atm (35 psi) without affecting its operability.

In an aspect the extraction process employed herein uses the ionic affinity of Nickel-60 or -61 or -64 to a solvent employed as a liquid mobile phase carrier to selectively remove the Nickel-60 or -61 or -64 from a liquid composition containing Nickel-60 or -61 or -64 and Copper-60 or -61 or -64, wherein the composition is loaded on a packing in a separation zone and the mobile phase carrier is passed therethrough.

In an aspect the term “stationary phase” refers to solid support such as packing including ion exchange resin contained within the lumen or interior of the chromatographic separation such as in a column over which or through which the mobile phase flows. The mobile phase may be continuous, semi-continuous or batch.

In an aspect the composition containing Nickel-60 or -61 or -64 in Copper-60 or -61 or -64 is typically a liquid and is injected into the mobile phase (HCl) of the chromatographic column through a coupled injector leaktight port. As the composition to be refined/purified flows with the mobile phase through the stationary phase in the chromatographic separation zone of the column, the components of that composition to be refined migrate to the stationary phase.

The main requisite for selection of a mobile phase herein is its capability to dissolve the composition containing the copper and nickel radionuclides at least up to a concentration suitable for the detection system coupled to the effluent of the column. This means that the column is selected to have the capability to provide the desired degree of refining/purification/extraction of the composition loaded onto the column so as to provide a refined Copper-60 or -61 or -64 radionuclide from a mixture of Copper-60 or -61 or -64 radionuclide and Nickel-60 or -61 or -64.

Basically this inventive process comprises admixing a portion of a multi-component composition to be refined (i.e. having a Nickel-64 component and desiring to be purified) with a first mobile phase carrier to form a chromatographically separable multi-component separable composition comprising a first mobile phase carrier. The first mobile phase carrier has a high affinity for the Nickel-64 which is the material to be separated form the Copper-64. The chromatographically separable composition is passed into a chromatographic separation zone having as packing therein ion exchange resins having an average particle diameter in the range from about 100 to about 200 microns. An eluent is thereby formed of a component (Nickel-64) of the multi-component composition. In an aspect the eluent is removed from the column and passed through an appropriate detector for analysis

In an aspect the temperature of the chromatographic column is in the range from about ambient temperature to about 60° or about 70° C. The initial addition of mobile phase carrier is at about 98° C. and subsequent additions are at about room temperature (about 25° C.).

In an aspect the eluent of the individual desired (Copper-60, or Copper-61, or Copper-64) radionuclide is temporarily retained within the chromatographic system. A second mobile phase carrier having an affinity for the temporarily retained copper radionuclide is passed/loaded into the chromatographic separation zone following a first mobile phase carrier, thereby forming a purified eluent containing the component of interest in a purified or refined form.

In an aspect the column is a HCl (hydrochloric acid) acid attack resistant plastic or glass construction or a suitable rounded container and has leakproof secure fittings at the ends of the column that connects the column to the injector at the loading end of the column and a detector at the effluent end. In an aspect the column has suitable internal configuration to hold the packing.

In an aspect the purified eluent comprising the purified copper radionuclide is thereafter passed into a label process for appropriate labeling of the refined copper radionuclide with a ligand, if desired.

In an aspect (aqueous) HCl is employed as a first mobile phase carrier. In an aspect the concentration of the HCl employed as a first mobile phase carrier to remove nickel radionuclide from the column is in the range from about 5 to about 7 and preferably from about 5.5 to about 6.5 molar. 6M HCl is prepared from concentrated 12 M, ultra pure 99.999999%, copper-free HCl and 18 Meg-ohm water. HCl (hydrochloric acid) also known as muriatic acid and chlorohydric acid is available commercially as an aqueous concentrate comprising about 12M.

In an aspect (aqueous) HCl is employed as a second mobile phase carrier to remove the temporarily intentionally retained copper radionuclide from the column. The concentration of the HCl employed as a liquid, a second mobile phase carrier, is in the range from about 0.3 to about 0.7 and preferably from about 0.4 to about 0.6 molar. 0.5M HCl is prepared from concentrated 12 M, ultra pure 99.999999%, copper-free HCl and 18 Meg-ohm water.

Basically the first mobile phase carrier is a high molarity aqueous hydrochloric acid composition and the second mobile phase carrier is a low molarity aqueous hydrochloric acid composition.

In an aspect the second mobile phase carrier is passed through the column after the passage of the first mobile phase carrier through the column. In an aspect both the first mobile phase carrier and second mobile phase carrier are passed through the column in the same direction over column packing.

Typical materials of construction of the first chromatographic separation zone include acid resistant plastic or glass such as plastics and glass resistant to chemical attack by 6N HCl (and above) and acid fumes or any suitable rounded container having a lumen therein.

In an aspect the removed eluent is further processed for ⁶⁰Ni, or ⁶¹Ni, or ⁶⁴Ni recovery recycling. In an aspect ⁶⁰Cu, or ⁶¹Cu, or ⁶⁴Cu is retained into the ion exchange column resin. The enriched nickel nuclide is eluted from the column and isolated for recycling purposes for the preparation on another target material. ⁶⁰Cu, or ⁶¹Cu, or ⁶⁴Cu is subsequently recovered by addition of about 0.5N HCl to elute purified ⁶⁰Cu, or ⁶¹ Cu, or ⁶⁴Cu for recovery and subsequently for labeling.

In an aspect from a safety perspective the process is monitored by using a suitable radiation detector and alerting system behind the column to monitor the activity displacement from the dissolution used to the recovery unit configured for, adapted to and affixed to the effluent process connection of the separation column.

### DETAILED DESCRIPTION OF THE DRAWINGS

FIG. 1 and FIG. 2 comprise schematic block diagrams of an illustrative functional chromatographic separation system for copper nuclear radionuclides employing therein the inventive process and apparatus of this discovery.

FIG. 1 is an illustrative process schematic which depicts a novel copper radionuclide separation and purification unit schematic.

The irradiated target produced by bombardment using a charged-particle accelerator lands in dissolution vessel 20 which is made of a material resistant to hot concentrated hydrochloric acid. In an aspect the target is said to land because it can be placed manually in dissolution vessel or placed there using the pneumatic target transfer line.

Reflux condenser 21 is placed on top the dissolution vessel 20 to keep the concentration of the acid at about 6M by condensing the hydrochloric acid fumes. In an aspect a single target is irradiated at a time using a cyclotron.

Anion exchange column 21 packed with resin sits near the dissolution vessel 20 to collect the target solution and to perform the separation and purification of Cu.

Empty syringe 23 actuated by a double-acting linear motion cylinder serves as pull and push of the dissolved target solution and rinse solution from the dissolution vessel 20 to anion exchange column 22. (Rinse solution is mobile phase carrier)

Pre-filled syringe 24 actuated by a single acting linear motion cylinder permits the first load of concentrated acid as the first mobile phase carrier to be heated into dissolution vessel 20, the rinse of dissolution vessel 20 and the purification of the Cu onto the anion exchange column 22.

Ni recovery vessel 25 retrieves eluents (first mobile phase carrier) containing mostly radioactive Ni ions. Eluents from passing the dissolved target solution and the purification of Cu from the anion exchange column 22.

Syringe 26 actuated by a single-acting linear motion cylinder pushes the low concentration hydrochloric acid onto the anion exchange column 22 to elute off the purified Cu which is collected into the final Cu recovery 27 or directed toward the line 28 going to the labeling unit.

Activity monitor 27 located behind dissolution vessel 20 records activity at the beginning. Activity monitor 28 located behind the anion exchange column 22 records activity during separation and purification stages. Activity monitor 29 located behind the purified Cu recovery 27 records activity at the end of the separation and purification process of Cu.

The symbol for the valves is a circle with a diagonal line crossing the center. There are digits from 1 to 9 next to each symbol on FIG. 1 (separation) and digits from 1 to 11 on FIG. 2 (labeling).

Throughout the separation and purification of Cu process nine actuated pinch valves are located at different sites to guide the path of the different solutions at different locations.

This separation and purification unit is contained within shielded box 30. In an aspect the separation and purification system is automated and designed for remote separation and purification of the ⁶⁰Cu, ⁶¹Cu and ⁶⁴Cu radionuclides of interest.

FIG. 2 is a schematic which shows the copper radionuclide unit labeling schematic. In an aspect the labeling unit is built with the substantially same components as for the separation unit. Pneumatic pinch valves are used to control the distribution of solution where it is desired. Linear motion actuators (pneumatic air cylinder) and vacuum are used along with syringes to dispense reagents at different stage of the labeling process and to collect the finale sterile labeled Cu. Solenoid valves are used to control the air cylinder plunger direction and to control the pinch valve actuation. Flow controllers are used to control speed of actuation. Solenoid valves are mounted on D-sub valve manifold thus minimizing space and making troubleshooting easier. In an aspect the labeling system is preferably automated and designed for remote labeling with a ligand of the purified radionuclide Copper-60 or, Copper-61 or, Copper-64 of interest.

In an aspect purified ⁶⁰Cu, or ⁶¹Cu, or ⁶⁴Cu is obtained in a form of copper chloride from the aforedescribed automated separation unit. The purified solution is loaded onto Alltech concentrating cartridge, or any other brand equivalent concentrating cartridges, and ⁶⁰Cu, or ⁶¹Cu, or ⁶⁴Cu is eluted off this concentrating cartridge with about 1-mL of 3N HCl.

In an aspect ⁶⁰Cu, or ⁶¹Cu, or ⁶⁴Cu-diacetyl-bis (N-methylthiosemicarbazone)—also denoted as Cu-ATSM is prepared by dissolving ATSM diacetyl-bis (N-methylthiosemicarbazone) into 1 mL DMSO (dimethyl sulfoxide). 10 μL of the ATSM ligand is added into a simple reaction line along with the eluant of ⁶⁰Cu, or ⁶¹Cu, or ⁶⁴Cu. ATSM ligand and ⁶⁰Cu, or ⁶¹Cu, or ⁶⁴Cu labeled by contact due to their inherent chemical kinetics. The ⁶⁰Cu, or ⁶¹Cu, or ⁶⁴Cu-ATSM solution is transferred onto a prewashed C₁₈ SepPak Light Cartridge. Sterile water is added onto the Sep-Pak® cartridge to wash out any free Cu and excess of Cl⁻ ions and other impurities (Co). This wash is collected into a waste recovery vessel. The ⁶⁰Cu, or ⁶¹Cu, or ⁶⁴Cu-ATSM solution is eluted off the SepPak with absolute ethanol (Ethyl alcohol-200 proof dehydrated alcohol U.S.P. Punctilious®). The final product is filtered through a 0.21 μm sterile filter prior to be loaded into a sterile syringe. (Sep-Pak® cartridge comprising a cartridge containing a silica-based bonded phase with strong hydrophobicity available from Waters, 34 Maple Street, Milford, Mass. 01757 USA. Sep-Pak® is a registered trademark of Waters.) Any suitable techniques for purification can also be employed.

The aforementioned automated chromatographic method and apparatus can be effectively utilized to provide an increased capacity for the multi-component copper-60/nickel-60 composition, or copper-61/nickel-61 composition, or copper-64/nickel-64 composition.

Irradiated ^{60, 61, 64}Cu Radionuclide Mixture Preparation

More in detail, in an aspect an irradiated isotopically enriched ⁶⁰Ni or ⁶¹Ni or ⁶⁴Ni material is prepared as a feed stream to a separation unit comprising illustratively as a separation unit an ion exchange column to prepare purified irradiated ⁶⁰Cu or ⁶¹Cu or ⁶⁴Cu respectively.

In an aspect isotopically enriched ⁶⁰Ni or ⁶¹Ni or ⁶⁴Ni is prepared and is plated (electrodeposited) onto a gold disk and irradiated with a proton beam to produce ⁶⁰Cu or ⁶¹Cu or ⁶⁴Cu via the ⁶⁰Ni(p,n)⁶⁰Cu or ⁶⁰Ni(p,n)⁶¹Cu or ⁶⁴Ni(p,n)⁶⁴Cu nuclear reaction respectively. In an aspect, electrodeposition of a nickel layer on a gold disk is carried out using an electrolytic cell comprising a reservoir made of Pyrex containing the electrolytic solution, a support plate, a Teflon® spacer to shape the deposition into a circle, and a graphite anode rod mounted in the center of the cell. A miniature electric motor having rotational servomechanism means rotates the rod during electrodeposition on the target in order to agitate the solution thus maintaining a flow of fresh electrolyte at the gold disk surface. A constant voltage is maintained to the electrolytic cell during the aforementioned plating process thus providing an electroplated ^{60 or 61 or 64}Ni target material for the production of ^{60 or 61 or 64}Cu.

In an aspect the preparation of ⁶⁴Cu and other copper radionuclides suitable for subsequent processing using a charged-particle accelerator is carried out using a process system substantially according to the disclosure in U.S. Pat. No. 6,011,825 hereinafter ('825 patent) which issued to Michael J. Welch, et al. on Jan. 4, 2000. The patent discloses a method for producing a radionuclide from a target nuclide expressing using an accelerator capable of generating a beam of charged particles at energies of at least about 5 MeV. A solid target which includes the target nuclide is loaded in a target holder suitable for use with the accelerator, and irradiated with the charged-particle beam at energies of at least about 5 MeV to form the radionuclide. After irradiation, the irradiated target is remotely and automatically transferred, without direct human contact and without human exposure to measurable ionizing radiation, from the target holder to an automated separation system. The irradiated target is transferred alone, in its own free form, without transferring any subassembly of the target holder. The radionuclide is then separated from unreacted target nuclide using the automatic and remotely operable separation system.

In a variation of this method, the irradiated target is transferred from the target holder to a pneumatic or hydraulic conveyance system which includes a transfer fluid moving through a transfer line, the fluid movement being effected by a motive force means. The irradiated target is conveyed using the pneumatic or hydraulic conveyance system, either in direct contact with the transfer and being entrained therein, or alternatively, in a transfer capsule which houses the target.

A preferred target comprises a substrate having a back surface and a front surface substantially parallel to and opposing the back surface. A target layer having an exposed surface is formed over the front surface of the substrate. In a preferred embodiment, the target layer covers a portion of the substrate surface, such that an edge margin of the substrate surface remains uncovered.

The target layer comprises a target material that comprises a target nuclide capable of reacting with charged particles having energies ranging from about 5 MeV to about 25 MeV to form radionuclides suitable for use in diagnostic or therapeutic radiopharmaceuticals. ⁶⁰Ni is a preferred target nuclide for producing ⁶⁰Cu, ⁶¹Ni is a preferred target nuclide for producing ⁶¹Cu and ⁶⁴Ni is a preferred target nuclide for producing ⁶⁴Cu.

The target material is preferably as isotopically pure as commercially possible with respect to the target nuclide. Isotopic purity of the target material impacts the production yield of the reaction. Target nuclides which are not naturally available in high concentrations are preferably isotopically enriched, While the degree of enrichment achievable and commercially available will vary depending on the target isotope, the target material preferably comprises at least about 75% target nuclide by weight, more preferably at least about 90% by weight, and most preferably at least about 95% by weight. For ⁶⁴Cu production, the ⁶⁴Ni is preferably at least about 95% enriched and more preferably at least about 98% enriched. The isotopic composition of commercially available 95% enriched ⁶⁴Ni is representative of enriched ⁶⁴Ni generally: 2.6% ⁵⁸Ni, 1.72% ⁶⁰Ni, 0.15% ⁶¹Ni, 0.53% ⁶²Ni, and 95(±0.3%) ⁶⁴Ni.

The target material is also preferably as chemically pure as commercially possible. The use of a target material that has a minimal amount of chemical impurities facilitates subsequent isolation and purification of the radionuclide of interest. The degree of chemical purity achievable and as commercially available will generally vary depending on the target nuclide being used and the impurity of concern. To produce radionuclides having a high specific activity, it is especially preferred that the target material have a minimal amount of carrier impurities and/or other chemical impurities which are difficult to separate from the product radionuclide. The level of carrier impurities in the target material is preferably low enough to allow production of the radionuclide at specific activities sufficient for clinical use in a radiopharmaceutical imaging composition or in a radiopharmaceutical therapeutic composition. Commercially available ⁶⁴Ni typically comprises natural copper carrier at a concentration of about 180 ppm by weight. ⁶⁴Cu having a specific activity suitable for diagnostic and therapeutic applications was produced using such commercially available ⁶⁴Ni target material. To achieve higher specific activities generally, the amount of carrier impurity present in commercially available target material is preferably reduced, for example, by purifying the target material prior to use in forming the target layer over the substrate surface. For ⁶⁴Cu production, carrier copper is so preferably separated from the enriched nickel target material using the ionic exchange method discussed below for separating ⁶⁴Cu produced by the present invention from unreacted ⁶⁴Ni target nuclide; or for separating ⁶¹Cu produced by the present invention from unreacted ⁶¹Ni target nuclide; or for separating ⁶⁰Cu produced by the present invention from unreacted ⁶⁰Ni target nuclide.

The substrate comprises a substrate material which is preferably chemically inert and capable of being separated from the target material and from the radionuclides produced during subsequent irradiation. The substrate material preferably has a melting point and a thermal conductivity which is at least about equal to the melting point and the thermal conductivity of the target material, respectively. Gold and platinum are preferred substrate materials. While the exact configuration (e.g. shape, thickness, etc.) of the substrate is not narrowly critical, the substrate is preferably shaped to facilitate use in a particular target holder and preferably thick enough to provide adequate support to the target layer during irradiation. For use with the target holder of the present invention, the substrate is preferably disc-shaped with diameters ranging from about 1.7 cm to about 2.3 cm and thicknesses ranging from about 0.4 mm to about 1 mm. The substrate most preferably has a diameter of 2 cm and a thickness of 1 mm.

In an aspect, a target is positioned in the anticipated charged-particle beam path of a low or medium energy accelerator by loading the target into a target bolder adapted for use with the accelerator. While the target described above is a preferred target, the target holder can be adapted to accommodate other target designs, For example, where the target material being irradiated is available in isotopically pure form, has adequate strength and is not prohibitively expensive, the target can consist completely of the target material without a supporting substrate. The target is preferably aligned with the anticipated beam path such that the entire beam cross-section impinges the target layer. Alignment is particularly preferred where the target area and the anticipated impingement area are matched.

The electroplating provides a suitable Nickel target material that after proton irradiation provides the desired Copper radionuclide.

However with regard to subsequent process separation, recovery and labeling steps a different process of the irradiated ^{60, 61, 64}Cu material is employed according to applicants discovery which is hereinafter more particularly described.

A unique process is employed for the separation of the irradiated ^{60, 61, 64}Cu prepared.

This specification and claims makes clear structure of a computer such as a PCC, or computer component implemented in either hardware or software and its associated hardware platform. The use of a computer is satisfied with at least one of—a programmed computer, a PLC programmed with the desired sequences of this discovery, with functionality implemented in hardware or hardware and software such as that; corresponding to the sequence in Tables I and II—a logic circuit or other component of a programmed computer that performs a series of specifically identified operations dictated by a computer program; and/or—a computer memory encoded with executable instructions representing a computer program that can cause a computer to function in a particular fashion.

Preparation of the Copper (⁶⁰Cu, ⁶¹Cu and ⁶⁴Cu) Separation and Purification Unit.

Illustratively to assemble, one begins by assembling the necessary supplies and preparing the (⁶⁰Cu, ⁶¹Cu and ⁶⁴Cu) copper separation and purification unit by weighing between 2.74 to 2.76 grams and preferably near 2.75 grams of packing material. Then adding about 5 mL of 6M HCl and gently stirring until slurry is formed. Then transferring resin slurry to ion exchange column and transferring an additional 20 mL of 6M HCl to column, rinsing sides of column during transfer, and allowing drainage via gravity into a collecting vessel. Once column is drained which gives approximately 4 cm of packing material or resin in the column, disposing accordingly of the 6M HCl. Then placing the pre-conditioned column onto the column holder on the copper separation and purification unit.

During irradiation of the disk, fill the dissolution vessel with 6-mL of 6N HCl dispensed from the prefilled 20-mL syringe (injection member). This acid is heated until it reaches about 98° C. at which point the temperature is maintained. After irradiation, place the irradiated disk in the heated 98° C. 6N HCl and dissolve the irradiated ⁶⁰Ni, ⁶¹Ni and ⁶⁴Ni off the gold disk for approximately 20 minutes. This hot target solution (temperature of about 98° C.) is loaded onto previously conditioned ion exchange column (pre-treated with 6N HCl) and the eluent, which contains enriched Ni ions, is recovered for further Ni recycling to make targets. ^{60, 61, 64}Cu is temporarily trapped/retained in the ion exchange column resin. To recover as much as possible any such ^{60, 61, 64}Cu activity, load a second volume of 6-mL of 6N HCl (dispensed from the same prefilled 20-mL syringe) into the dissolution vessel to rinse out any remaining activity. Pull and load this 6N HCl rinse (mobile phase carrier) into the empty 20-mL syringe and load it onto the anion exchange column and recovered this second eluent fraction for Ni recycling to make Ni targets.

Purification—Purifying the ⁶⁴Cu follows the aforedescribed novel automated separation process.

To purify ⁶⁴Cu, load a third volume of 6-mL of 6N HCl (dispensed from the same prefilled 20-mL syringe) onto the column and recovered this third eluant fraction for Ni recycling. Finally, load onto the column 8-mL of 0.5N HCl (second mobile phase carrier) to elute off the ^{60 or 61 or 64}Cu ions. This finale eluent contains the ^{60 or 61 or 64}Cu in approximately 8-mL of 0.5N HCl. This can be distributed for further research uses or it can be directed into a second unit for labeling purpose. During this whole dissolution, separation and purification steps, activity displacement is monitored at strategic locations such as the dissolution vessel, the ion exchange column and the finale recovery vessel.

In an aspect the discovery further comprises a labeling process and unit for the highly purified copper radionuclide (⁶⁰Cu, ⁶¹Cu and ⁶⁴Cu).

Preparation of the copper (⁶⁰Cu, ⁶¹Cu and ⁶⁴Cu) labeling unit is an important aspect of this discovery.

One begins by assembling the necessary supplies and preparing the (⁶⁰Cu, ⁶¹Cu and ⁶⁴Cu) copper labeling unit by pre-conditioning the Alltech concentrating cartridge (available from Alltech Associates, 2051 Waukegan Road, Deerfield, Ill. 60015) with 3N and 0.5N HCl and the C₁₈ Sep-Pak cartridge with ethanol follow by sterile water. Pre-condition the Millipore sterile filter with 1.5-mL of sterile saline solution. Place an empty 10-mL sterile syringe onto the final collection clamp. Prefill a 15-μL sterile syringe with 10-μL of ligand solution and place it onto clamp. Prefill a 1-mL sterile syringe with 500-μL ethanol and place onto clamp. Pre-fill a second 1-mL sterile syringe with 1-mL of 3N HCl and place onto clamp. Pre-fill a 10-mL sterile syringe with 10-mL sterile saline solution and place onto clamp. Pre-fill another 10-mL sterile syringe with 10-mL of sterile water and place onto clamp. Finally, place a 25-mL waste recovery vial into place. (Sep-Pak® cartridge comprises a cartridge containing a silica-based bonded phase with strong hydrophobicity reportedly using trifunctional bonding chemistry available from Waters, 34 Maple Street, Milford, Mass. 01757 USA. Sep-Pak® is a registered trademark of Waters.)

Labeling of ^{60 or 61 or 64}Cu.

Once the ^{60 or 61 or 64}Cu is eluted off the aforedescribed anion exchange column, load the eluted second mobile phase carrier composition onto the Alltech concentrating cartridge and collect the 0.5N HCl into the waste recovery vial. The ^{60 or 61 or 64}Cu is loaded onto the concentrating cartridge. Push the 10-μL of ligand solution (containing ATSM) into the reaction line. At the same time elute the Alltech concentrating cartridge by loading on 1-mL of 3N HCl. The labeling occurs via chemical kinetics through the reaction line. Load the mixture onto the C₁₈ Sep-Pak cartridge and collect the waste solution into the recovery vial. The labeled product is trapped onto the Sep-Pak cartridge. Load 10-mL of sterile water into the reaction line and through the C₁₈ Sep-Pak cartridge and collect the waste into the waste recovery vial. This purifies the labeled compound. To elute the Sep-Pak, load 500-μL of ethanol through it and collect into the final sterile syringe. Finally, load 7-mL of sterile saline solution onto the C₁₈ Sep-Pak cartridge and sterile filter and add into the final sterile syringe. Activity displacement is monitored at strategic locations such as the concentrating cartridge, the Sep-Pak cartridge and the final syringe.

Labeling results confirmed the feasibility of using a simple reaction line for this synthesis. These two automated units (separation and purification unit and the labeling unit) are designed to be used independently or in an in-line arrangement with the separation and purification unit feeding the labeling unit.

The inventors favor the use of disposable accessories (syringes and tubing) to minimize cross contamination between processing and to minimize preparation time and to eliminate cleaning procedures. In an aspect new disposable accessories and reagents will be placed in the units before each process. The activity transfer between different steps will be monitored with radioactivity detectors placed at strategic locations. Linear motion actuators will be used with syringes to dispense reagents into the ion exchange column for each step during separation and purification. Vacuum and linear motion actuators will be used to transfer the reagents at different steps for the labeling. The final sterile copper radionuclide labeled compound will be collected into a sterile syringe for patient administration. A graphical user interface and a personal computer will be used to operate each unit and for keeping records.

In an aspect separation and purification is automated according to Table I attached (Control Sequence for Automated Cu Separation and Purification). An illustrated sequence for the automated control of the valves and syringes is shown in Table 1 and detailed below. Table I provides this illustrative sequence as a listing of numbered steps in the left hand column more particularly identified in another companion adjacent column which recites a function of the step.

Valves are numbered 1-9 and correspond to valves shown. Syringes 23, 24 and 25 are shown in the right hand column with the upward pointing arrows indicating pulling plunger and the downward pointing arrows indicating pushing plunger. In this control sequence for automated copper labeling steps are associated with valves, syringes, and vacuum according to recited elements of a row of the Table 1 in FIG. 1 of the process schematic for the separation and purification unit.

As used herein the terms “automate” and “automated” mean as applied to a process, a conversion to automation and to use the techniques of automation, i.e. automated teaching such as using a PLC with PLC software programmed to a desired sequence such as those in the Table I, Table II or Table I and Table II. The term “automation” includes a system or method in which many or all of the processes of production and movement and control (including the individual processes of separation, recovery and purification or any combination of those processes) are automatically controlled by self operating electronic, mechanical or electromechanical functional and functioning means. Such automation may be accomplished by the appropriate use of one or more computer or instructional units providing the implementing instruction to the process equipment. In an aspect to automate includes to operate or control by using a functioning computer or software equipped control system such as a computer or instruction unit configurably functionally loaded with a control sequence such as shown in Table I, Table II or Table I and Table II herein as instructions and suitable software interfaced with valves and syringes. In another aspect a functioning computer or software equipped instructional control system is electronically coupled to a valve and/or syringe which is controllable by electronic signal. In an aspect transducers are employed to provide means of effective communication between the instruction unit and the valves and/or syringes. The process outlined herein is for illustration purposes only and no dimension provided herein is deemed to be limiting in any way.

It is understood that appropriate size valves, cylinders, piping and various process connections providing operability will be made after reading this specification, claims and drawings. It is also understood that elements of this process including equipment, software, computer, valves, piping, tubing, column, packing and connections are functionally capable enabled i.e. they are connected in a manner so as to make the process operable for its intended purpose and objective(s).

In an aspect automation of a process is accomplished by utilizing an electronic control system wherein a ladder logic software program is written and utilized to instruct timers, counters, and motion controllers to a specific sequence. Typically the program provides analog signals and analog outputs wherein such analog signals and such analog outputs are used to instruct the temperature sequence and to monitor activity throughout processing. The electric/pneumatic power/energy is supplied as needed to enable the process to be operable and functional.

In this automated process each species of radioactive copper is individually processed, prepared and recovered (ie., individually, separately and respectively). For example, ⁶⁰Cu is processed, prepared and recovered, ⁶¹Cu is processed, prepared and recovered and ⁶⁴Cu is processed, prepared and recovered in this discovery.

In an aspect, Ladder Logic is the main programming method used for a PLC and has been developed to mimic relay logic. Ladder logic programming looks like a ladder or a flow chart. Illustratively there are two vertical lines coming down the programming environment, one on the left and one on the right; then there are rungs of conditionals on the left that lead to outputs on the right. In Ladder logic programming there are registers which are of four kinds, X's that are inputs, Y's that are outputs, D's that are data that can form integer, hexadecimal and real numbers, and R's that are internal relays.

The term “valves” as used herein includes any flow control apparatus which is configureably designed to maintain, restrict, or meter the flow of materials through pipes, hoses, tubing or entire systems such as of the novel chromatograph and labeling system. Valves typically allow flow in an open position and when closed restrict or shutoff flow.

In an aspect solenoid valves includes any electromechanical device that uses a solenoid to control valve actuation. Electrical current such as that from a computer or electrical instructional unit is configureably and operably supplied and connected to the solenoid coil of the solenoid valve. Resulting in a magnetic field which acts upon a plunger in turn, whose resulting motion actuates its associated valve resulting in opening and closing thereof. Like computers solenoid valves are activated by supplying electrical power to the solenoid.

As used herein, the term “syringe” is representative of an injection member.

In an aspect an illustrative labeling process is automated according to the control sequence shown for automated copper labeling in Table II attached. An illustrative sequence for the automated control of the valves and plungers (syringes) shown in FIG. 2 is summarized in Table II and detailed below.

More in detail, steps numbered 12-17 are recited in the left hand column and are associated with an action in the process schematic of Table I. In similar fashion as for previously described Table 1, valves are identified in this Table 2 and the position of the valve as open or shut are respectively presented in Table 2. An open valve is denoted as “o” (for open) and a valve close is denoted as “x”. Syringes 32, 33, 37, 38, 39 and 42 are denoted in an adjacent right hand column with upward pointing arrows ↑ indicating pulling plunger of the syringe and the downward pointing arrows ↓ indicating pushing plunger of the syringe. The application of vac (vacuum) is shown in the right hand column of Table 2 with the symbol “o” in that column of that Table denoting application of On vacuum and the symbol “x” in that column of that Table denoting application of Off vacuum. In this control sequence for automated copper labeling steps are associated with valves, syringes and vacuum according to recited elements of a row of the Table 2.

In Tables 1 and 2, “x” and “o” are convenient alphabetical symbols with “o” representative of an open position of a valve (flow permitted therethrough as valve is open) and “x” a symbol of a closed valve (no flow permitted therethrough as valve is closed).

One practicing this discovery will understand that the process is monitored by a capable radiation detector at different locations of the automated process which monitors the displacement of radioactivity and also indicates the ratio of product activity being separated. From this detector, any adjustment may be made to the process if necessary. All electrical poer and pneumatic poer is made available to the automatic process system to enable a capable successful carrying out of this discovery and recovery of the desired product.

In an aspect an automatically labeled ⁶⁰Cu labeled ligand is utilized to treat a living mammal cancer patient. Various aspects of that treatment are now presented.

It is understood that ⁶⁰Cu, ⁶¹Cu and ⁶⁴Cu are used in diagnosis and ⁶¹Cu and ⁶⁴Cu are used in therapies and that those products herein are ready for there respective use.

It is understood that suitable temperatures, pressure, mole ratios and other operating conditions are such that a suitable complexing reaction occurs resulting in the production of an radionuclide coupled with a pharmacologically acceptable ligand.

In an aspect a functional emitting copper radiolabeled material (tracer compound(s)) of this discovery is administered parenterally, i.e., by intravenous, i.p., intrathecal or enteral administration to a living mammal patient. In an aspect the radiolabeled emits a functional externally image detectable amount of desired radioactivity in the mammal. In a medical aspect the amount of emitted radioactivity is an amount which imparts a diagnostic or therapeutic benefit to the mammalian patient having cancer. In an aspect a therapeutic benefit is that benefit which is medicinally and therapeutically beneficial to the living mammalian afflicted with cancer. In an aspect a cytotoxic amount is an effective lethal amount of a therapeutic compound which beneficially kills or retards cancer cells. Useful radiochemical methods are found in the textbook INSTRUMENTAL METHODS OF ANALYSIS, Willard, Hobart H.; Merritt, Jr., Lynne L.; and Dean, John A., 4^{th} Edition, D. Van Nostrand Company, Inc. August 1965

A hot cell is a closed work area in which radioactive materials may be manipulated without exposing the operator to significant or unacceptable amounts of radiation. Some cells are dedicated to the production of a single radioisotope in order to minimize contamination. Other cells are used to process a wide range of nuclides while still others are used for storage and transfer functions. They are an integral part of radioactive nuclide production and their care and maintenance are high priorities.

It is understood after reading the specification and claims that one practicing this discovery in a radioactive environment will use all necessary and practical safety protective equipment including the use of all personal radioactive protection gear.

In an aspect a detectably labeled copper ligand (tracer compound and a copper isotope prepared and recovered herein) is effectively administered to a mammal or to a biological sample thereof or there from and the sample is analyzed and a diagnosis is made or obtained. In an aspect, a biological sample of the mammal comprises a representative sample taken of at least one of blood, vessels, atheroma, liver, and other body tissues a well as biopsies of body organs such as a liver biopsy or a muscle biopsy of a living mammal. In an aspect, the amount of biological sample is that amount or volume which is sufficient to provide for an analysis. In an aspect this discovery is employed in small animal imaging.

As used herein, the term “biological sample” or “biologic sample” includes a sample of a suitable size of a living mammal such as a sample of size and composition suitable to use in the methods disclosed herein.

In an aspect a pharmaceutical composition is automatically prepared in a process comprising automatically separating and purifying copper-60 or copper-61 or copper-64 employing a process following the schematic of FIG. 1 and configured to accommodate and utilize the automatic sequence of separation and purification illustrated in Table I. In an aspect the pharmaceutical composition comprises at least one copper labeled compound as a (⁶⁰Cu, ⁶¹Cu and ⁶⁴Cu) radiolabeled compound.

The product nuclide such as the purified copper-60, copper-61 or copper-64 is recovered from the process producing that product nuclide and readied for use in diagnostic imagining as is described herein which in an aspect includes the use of the product nuclide in a pharmaceutical composition which is effectively administered to a patient such as a living mammal such as a human.

At the end of the automated processing, a purified nuclide (⁶⁰Cu, ⁶¹Cu and ⁶⁴Cu) is ready for distribution to researchers or directed into the next automated unit for labeling with ATSM ligand. All processing and chemistry are scheduled according to the time of use or injection for PET studies and diagnostics for example.

At the end of the automated separation and purification process a purified nuclide (⁶⁰Cu, ⁶¹Cu and ⁶⁴Cu) is contained within a closed container which is aliquoted and shielded until chemistry processing, or at the end of the automated labeling unit a purified ATSM labeled copper is contained within a sterile Norm-Ject® syringe which is shielded until its use.

In an aspect a mammal host selected from at least one of a living human and non human animal such as canine, feline, equestrian, murine including dogs, cats, rabbits, guinea pigs, hamsters, mice, rats, horses, goats, deer, sheep, rodents, pigs and cows. In an aspect a veterinarian treats a dog having cancer. In an aspect the mammal host is a patient. In an aspect this discovery is employed in small animal imaging.

In an aspect, depending on its form, the administered formulation is suitably formulated for ease of facilitation of administration and use by the mammal patient and may contain a binder, disintegrating agent, lubricant, sweetener, a liquid carrier.

In an aspect a copper radiolabeled compound is administered to a living mammal as a pharmacologically acceptable composition such as solutions of a labeled compound or its salts can be prepared in water, optionally mixed with a nontoxic surfactant or saline may be employed.

In an aspect the amount of time elapsing between imaging is a time which provides for a useful and meaningful comparison of acquired images.

Accordingly, the discovery includes a pharmaceutical composition comprising a labeled compound as described hereinabove; or a pharmaceutically acceptable salt thereof; and a pharmaceutically acceptable diluent or carrier.

In an aspect a therapeutic rate titration is performed wherein the living mammalian afflicted with cancer is administered a series of dosages and respective effects therefrom or thereafter are determined by an inventive method herein at respective dosages and times. In this manner a therapeutic dosage curve or titration is obtained for determining dosage for that mammal patient.

Successful and effective administration may be performed by local or systemic application as appropriate. Administration of compositions may be done by inhalation, orally, rectally or parenterally, such as by intramuscular, subcutaneous, intraarticular, intracranial, intradermal, intraocular, intraperitoneal, intrathecal and intravenous injection.

PET or Positron Emission Tomography, (including microPET) is a non-invasive molecular diagnostic imaging (standard) medical procedure that produce (i.e. capture and optionally record) multiple acquisitions i.e. images of the body's biological functions and in an aspect are used to determine the extent of malignant disease. In an aspect, these imaging procedures show the presence and distribution of a radiolabeled detectable functionally emitting radiolabeled chemical i.e. a radionuclide acquisitioned at various selected times. Advantageously these two imaging procedures depict metabolic characteristics of tissues and changes therein.

In an aspect, data acquisition and detection using positron emission tomography (PET imaging) comprises detection of energy emitted from (⁶⁰Cu, ⁶¹Cu and ⁶⁴Cu) radionuclides that decay and are located within a mammalian patient's body. In an aspect this is possible by virtue of administration of a radiolabeled peptide compound to the patient.

A useful text on PET is clinical positive emission tomography, Gustav K. Schulthess, Lipcott, Williams & Williams 2000.

MicroPet® is also useful in this diagnostic imaging (MicroPET® is a dedicated PET scanner designed for high resolution imaging of small laboratory animals. One such scanner is available from Concorde Microsystems, Inc. 10427 Cogdill Rd, Suite 500 Knoxyille, Tenn. 37932 USA). Other manufacturers also offers other small animal scanner for example Mosaic® from Philips (Andover, Mass. 01810, USA.

In an aspect images are taken over elapsed time in dynamic fashion to assemble a developing or developed scenario of situations in the living mammalian patient. The location of the (⁶⁰Cu, ⁶¹Cu and ⁶⁴Cu) radioactivity detected by the detector is indicative of the location of the cancer.

In an aspect a PET image is taken of a mammal after administration of a compound to the mammal.

After the (⁶⁰Cu, ⁶¹Cu and ⁶⁴Cu) radiolabeled copper compound is administered to a patient, the (⁶⁰Cu, ⁶¹Cu and ⁶⁴Cu) radioactivity travels through the body and localizes in the appropriate areas of the body and is detected by detection and data image acquisitions of the PET scanner.

Typically an adequate amount of time is allowed to pass for the treated mammal to come to an equilibrium state following satisfactory administration of the (⁶⁰Cu, ⁶¹Cu and ⁶⁴Cu) radioligand to the mammal. Typically the mammal is placed in a position near the PET instrument allowing satisfactory operation of the PET instrument. The PET instrument is equipped with all necessary operable software and operation requirements.

Generally after the mammal has received its effective administration of (⁶⁰Cu, ⁶¹Cu and ⁶⁴Cu) radiolabeled copper compound the mammal is taken to an examination room that houses the PET scanner, which has a opening in the middle. In the PET scanner there are multiple rings of detectors that record the emission of energy from the radioactive substance now within in the mammal. In an aspect the mammal is moved into the hole of the machine. The images are displayed on the monitor of a computer, suitably equipped and operably coupled to the PET scanner instrument for acquiring. In an aspect the image of emitted radioactivity of the mammal provides a location for the cancer in that mammalian patient with the theory being the that radioactive material being retained by the mammal indicates the presence and location of the cancer in the mammal that has received the (⁶⁰Cu, ⁶¹Cu and ⁶⁴Cu) radiolabled copper compound.

In an aspect an internal radiation cancer therapy useful on living mammals comprising administering anti-cancer compounds synthetically labeled with automatically prepared purified ⁶⁰Cu, or ⁶¹Cu, or ⁶⁴Cu to such living mammals. In an aspect a treatment of malignant neoplasm in living mammals (human and nonhuman) comprises administering anti-cancer compounds ligated with purified ⁶⁰Cu, or ⁶¹Cu, or ⁶⁴Cu labeled compounds.

In an aspect, a method for diagnosing a human mammal for the presence of a cancer or myocardial infarction or stroke comprises administering to the mammal a diagnostic imaging detectable effective amount of a purified ⁶⁰Cu, or ⁶¹Cu, or ⁶⁴Cu labeled compound, detecting binding of the at least one highly purified ⁶⁰Cu, or ⁶¹Cu, or ⁶⁴Cu labeled compound to a tumor in the mammal. In an aspect the method further comprises determining that a mammalian tumor is present in the mammal being administered to upon detecting binding, thus diagnosing the mammal. In an aspect the method comprises producing an acquisition of the detection of tumor in the administered to mammal. In an aspect detection of emitted radioactivity indicates the presence of and location of cancer in the mammal being diagnosed.

In an aspect, a marker for cancer comprises a purified ⁶⁰Cu, or ⁶¹Cu, or ⁶⁴Cu labeled compound prepared in accordance with this discovery having been recovered from the process and having an explicit provocative binding efficacy to a tumor in a living mammal.

In an aspect, a novel pharmaceutical composition comprises a novel purified ⁶⁰Cu, or ⁶¹Cu, or ⁶⁴Cu labeled compound prepared by the process of this discovery and a pharmaceutically acceptable diluent or carrier.

In an aspect, a pharmaceutical composition effective for treating human or non-human mammalian neoplastic disorder comprises a purified ⁶¹Cu or ⁶⁴Cu labeled compound in a composition including a pharmaceutically acceptable carrier.

In an aspect a pharmaceutically acceptable salt includes any water soluble salt which is pharmaceutically suitable to the mammalian recipient of the peptide or radiolabeled compound. In an aspect a pharmaceutically acceptable diluent or carrier includes an aqueous diluent or any diluent or carrier which is innocuous to the mammal recipient of the peptide compound and which provides for facilitation of the administration of the peptide compound(s) and their radionuclide counterparts.

The precise dosage of the detectably labeled peptide compound to be administered and the length of time over which administration is carried out will depend on a number of factors including the age and weight of the mammal patient and the route of administration.

In an aspect a therapeutic rate titration is performed wherein the living mammalian afflicted with cancer or believing to be so afflicted with cancer is administered a series of dosages and respective effects therefrom or thereafter are determined by an inventive method herein at respective dosages and times. In this manner a therapeutic dosage curve or titration is obtained for determining dosage for that mammal patient.

In an aspect, the radionuclide is purified and the compound administered to the animal is optionally pure or is purified.

Effective, administration may be performed by local or systemic application as appropriate. Administration of compositions may be done by inhalation, orally, rectally or parenterally, such as by intramuscular, subcutaneous, intraarticular, intracranial, intradermal, intraocular, intraperitoneal, intrathecal and intravenous injection. The injection may be by stereotaxic injection. Local administration may also be performed, e.g. at an affected site e.g. by use of a catheter or syringe. Treatment by topical application of a composition, e.g. an ointment, to the skin is appropriate. Administration may be performed at intervals of time, such as two or more applications, at some intervals, such as several times a day, or at periodic intervals of the daily or daily.

In an aspect, a method to determine the presence of a stroke or myocardial diseases or proliferative status of a cancer cell in a living mammal comprises administering to a living mammal afflicted with a malignant tumor, an effective amount of a purified ⁶⁰Cu, or ⁶¹Cu, or ⁶⁴Cu labeled compound and determining the extent to which the detectably-labeled isotopic copper compound binds to cells of a tumor in the treated mammal, the extent providing a measure of the proliferative status of the cancer cells in the treated mammal. In an aspect the living mammal is nonhuman. In an aspect determining the proliferative status includes assessing the proliferative status of a breast cancerous tumor. In an aspect ⁶⁰Cu, ⁶¹Cu and ⁶⁴Cu are the recovered products of this automated process.

In an aspect, a method for diagnostic imaging of a mammalian tissue comprises administering to the tissue of the mammal a diagnostic imaging amount of purified ⁶⁰Cu, or ⁶¹Cu, or ⁶⁴Cu labeled compound comprising a detectable amount of ⁶⁰Cu and detecting an image of that tissue. In an aspect the living mammal is nonhuman. In an aspect, the image is used to diagnose mammalian tissue.

In an aspect, a method for in vivo detection of a cancer cell in living mammalian tissue sample comprises contacting a mammalian tissue sample comprising a cell with an in vivo effective diagnostic imaging amount of at least one highly purified ⁶⁰Cu, ⁶¹Cu and ⁶⁴Cu labeled compound for a time and under conditions sufficient and effective for binding of highly purified ⁶⁰Cu, ⁶¹Cu and ⁶⁴Cu labeled compound to the cell and detecting such binding indicative of an association with the presence and location of cancer in the contacted cell. In an aspect the detecting is by image acquisition. In an aspect the highly purified ⁶⁰Cu, ⁶¹Cu and ⁶⁴Cu labeled compound is a tracer for cancer. In an aspect, the cell(s) is in a previously obtained biological sample from a mammal. In an aspect such binding is indicative of the presence of and location of a cancer cell. In an aspect, the mammal is a human and the radionuclide is ⁶⁰Cu. In an aspect the living mammal is nonhuman. In an aspect the extent of binding is determined by comparing the amount of radioactivity administered to the animal with the amount of radioactivity and location of radioactivity detected by image acquisition.

In an aspect, a method for determining proliferation and/or progression of a cancer as a disorder in a living mammal comprises administering to a living mammal a diagnostic imaging detectable amount of at least one highly purified ⁶⁰Cu, ⁶¹Cu and ⁶⁴Cu labeled compound at a first selected time, detecting an image of a tissue in the mammal being treated at a second selected (later) time respectively detecting an image of a tissue at both times, comparing the images and determining if the detected image at the later time is smaller than the detected image at the first time. In an aspect the elapsed time between the first time and second time is selected to be a time duration significant amount. In an aspect the living mammal is nonhuman. In an aspect the comparison is used to determine proliferation and/or progression of a cancer in a mammal.

In an aspect, a method for identifying a modulating effect (and regression effect) of a cancer in a living mammal with a disorder, comprises administering to the mammal a diagnostic imaging detectable amount of at least one highly purified ⁶⁰Cu, ⁶¹Cu and ⁶⁴Cu labeled compound at a first time, detecting and acquisitioning an image of a tissue in the mammal being treated, administering a highly purified ⁶⁰Cu, ⁶¹Cu and ⁶⁴Cu labeled compound to the mammal and at a second (later) time respectively detecting and acquisitioning an image of a tissue of the mammal being treated, comparing the respective images and determining that there has been an prophylactic effect and/or regression and/or modulation of the cancer. In an aspect the comparison shows the amount of regression over time. In an aspect the living mammal is nonhuman. In an aspect a comparison shows the prophylactic effect of the compound and its toxicity to cancer. In an aspect a comparison shows the efficacy of the compound to killing cancer in a living mammal.

PET (or “Positron Emission Tomography”) is a non-invasive molecular diagnostic imaging (standard) medical procedures that produce (i.e. capture and optionally record) multiple acquisitions i.e. images of the body's biological functions and in an aspect are used to determine the extent of malignant disease. In an aspect, these imaging procedures show the presence and distribution of a radiolabeled detectable functionally emitting radiolabeled chemical i.e. a radionuclide acquisitioned at various selected times. Advantageously these two imaging procedures depict both metabolic characteristics of tissues and changes therein.

In an aspect an external measurement is made of the two high energy photons emitted in opposite directions when a positron-emitting radionuclide decays in a patient. A large number of scintillation detectors detect these photon pairs and measure the sum of radioactivity along many different paths through the patient undergoing measurement. Appropriate software associated with the operating instrument reconstructs a three-dimensional image of the patient and the concentrations of radionuclides can be expressed in quantitative units of radiotracer concentration per ml of tissue.

In an aspect images are acquisitioned (taken) over elapsed time in dynamic fashion to assemble a developing or developed scenario of developing or changing situations in the mammalian patient. It is believed that the tumorous or cancerous areas have a higher density of these receptors than surrounding normal tissue and thus that is why such areas show up on the image.

In an aspect a PET image or microPET image is taken of a living mammal after administration of a cancer or tumor detector compound to a living mammal. The image may be retained in computer storage if desired. A number of images may be acquired as a function of elapsed time to produce a profile over time of the images.

In an aspect the compound with its radionuclide is administered to the patient as an aqueous composition such as a saline composition to the living animal such as to a human. Typically the compound and its radionuclide will be formulated as a water soluble salt and administered in an aqueous formulation comprising that water soluble salt of the compound and its radionuclide.

In an aspect a radioactive substance is produced in a process and is attached, or tagged, to a tracer compound known as radiolabeling. The tracer molecule can be either a complexing ligand or a biomolecule namely a peptide or engineered antibody. After this radiolabeled compound labeled with one of ⁶⁰Cu, ⁶¹Cu or ⁶⁴Cu is administered to a patient, radioactivity travels through the vascular circulator (blood) system of the body and localizes in the appropriate areas of the body and is detected by the PET scanner. For example a radiolabeled peptide will localize in areas where the specific receptor for the peptide is expressed.

In an aspect a tracer compound can be attached to a radionuclide by using a chelating group. Such chelating groups are well known in the art and include polycarboxylic acids such as for example diethylenetriaminepentaacetic acid, ethylenediaminetetraacetic acid, and the like, or analogs or homologs thereof, as well as the chelating groups disclosed in Anderson and Welch (Chem Rev. 99: 2219-2234, 1999) and Jurisson and Lydon (Chem. Rev. 99: 2205-2218, 1999).

The chelating group or the radionuclide therein may be attached directly to a compound or by means of a divalent or bifunctional organic linker group. Such bifunctional organic linker groups are well known in the art and are preferably less than about 50 angstroms in length. Examples of suitable bifunctional linker groups include 2-carboxymethyl, 3-carboxypropyl, 4-carboxybutyl, and the like. The linker group may also be attached at any synthetically feasible position.

Typically an adequate amount of time is allowed to lapse for the treated living mammal (i.e. having received the radiolabeled peptide) to come to an equilibrium state following satisfactory administration of the peptide radioligand to the mammal. Typically the mammal is placed in a position near the PET instrument or microPET® instrument allowing satisfactory operation of the PET instrument. The PET instruments are equipped with all necessary operably communicative instructional software and operation requirements.

Generally after mammal has received its administration of the radiolabeled peptide the mammal is placed in/on the PET scanner, which has a opening in the middle. In the PET scanner there are multiple rings of detectors that record the emission of energy from the radioactive substance now within in the mammal. In an aspect the mammal is comfortably moved into the hole of the machine. The images are displayed on the monitor of a computer, suitably equipped and operably coupled to the PET scanner instrument for acquiring.

### REFERENCES

1. McCarthy D W, Shefer R E, Klinkowstein R E, Bass L A, Margeneau W H, Cutler C S, Anderson C J, Welch M J. Nucl Med Biol 1997; 24: 35-43.
2. Lewis J S, McCarthy D W, McCarthy T J, Fujibayashi Y, Welch M J. J Nucl Med 1999; 40:177-183.
3. Dehdashti F, Grigsby R W, Mintun M A, Lewis J S, Siegel B A, Welch M J. Int J Radiation Oncology Biol Phys 2003; 55: 1233-1238.
4. Dehdashti F, Mintun M A, Lewis J S, Bradley J, Govindan R, Laforest R, Welch M J, Siegel B A. Eur J Nucl Med Mol Imaging 2003; 30:844-850
5. Fujibayashi Y, Cutler C S, Anderson C J, McCarthy D W, Jones L A, Sharp T, Yonekura Y, Welch M J. Nucl Med Biol 1999; 26: 117-121.

### EXAMPLES

A benchtop setup comprising a reaction line of non-DEHP (di-ethylhexyl phthalate (DEHP)) minibore tubing, commercial stopcock 3-way valves, cartridges, filter, different sized syringes and small air powered vacuum pup was assembled and tested. Vacuum force was significant enough to displace the fluids at required locations. Effective labeling of the activity with the ligand was obtained according to results from a Thin Layer Chromatography reading.

Advantageously this discovery provides a process for producing (⁶⁰Cu, ⁶¹Cu and ⁶⁴Cu) radionuclides in significant yields and at specific activities which are suitable for use in diagnostic and therapeutic applications. The discovery further allows for the recovery of enriched nickel isotopes (used to produce the copper isotopes) for recycling purposes. The discovery also provides a system/method in which such purified (⁶⁰Cu, ⁶¹Cu and ⁶⁴Cu) recovery is done automatically with minimal human intervention and therefore, without significant human exposure to ionizing radiation. In an option the process of discovery herein includes operation of the units for separating, purification and labeling of (⁶⁰Cu, ⁶¹Cu and ⁶⁴Cu) compounds by automated control systems.

Illustrative Materials Used

Hydrochloric Acid, High purity, 6.0M, prepared at Washington University in St. Louis, One Brookings Drive, St. Louis, Mo. 63110, USA.

Hydrochloric Acid, High purity, 3.0M, prepared at Washington University in St. Louis.

Hydrochloric Acid, High purity, 0.5M, prepared at Washington University in St. Louis.

⁽¹⁾ High purity HCl (99.9999%, 12.1 M) Alfa Aesar, Inc. or equivalent diluted with MilliQ water to desired concentration

ATSM, prepared at Washington University in St. Louis, One Brookings Drive, St. Louis, Mo. 63110, USA.

2,3-Butanedione, 97% (Aldrich Chemical Company Inc.) or equivalent

4-Methyl-3-thiosemicarbazide, 97% (Aldrich Chemical Company Inc.) or equivalent

Acetic acid, Glacial, 99.99% (Aldrich Chemical Company Inc.) or equivalent

Sodium acetate NaOAc, 1M, produced at Washington University in St. Louis, One Brookings Drive, St. Louis, Mo. 63110, USA

⁽²⁾ Sodium acetate (99.995%) Aldrich Chemical Company, Inc. or equivalent dissolved with MilliQ water to desired concentration

Ethanol, USP (Aaper, #92402 or equivalent)

Dimethyl Sulfoxide (DMSO) (Aldrich, #47,230-1 or equivalent)

Saline (0.9% 1M Sodium Chloride for Injection), sterile (American pharmaceutical partners, #NDC63323-186-10 or equivalent)

Sterile water for injection (American Pharmaceutical Partners, #NDC63323-185-20 or equivalent)

Sep-Pak C-18 filter cartridge (Waters # WAT023501) or equivalent

Maxi-Clean IC-H cartridge (Alltech # 30264) or equivalent

Millipore™ sterile filters, 0.22 μm pore (Millipore, millex gs 20 μm) or equivalent

Syringes (Norm-Ject®) (Air-Tite Products Co, Inc.) or equivalent

Air Cylinders SMC series CG1 (single and double acting, 20 mm bore) or equivalent

Air Cylinders SMC series NCG (3-position tandem, 20 mm bore) or equivalent

Solenoid valves, SMC series SY3000 (5-ports, 24VDC, plug-in) or equivalent

Valves manifold, SMC series SS5Y3 (D-Sub) or equivalent

Flow controllers SMC series AS or equivalent

Pinch valves, SMC series XT34 or equivalent

Solenoid valves, SMC series SYJ500 (3-ports, 24VDC) or equivalent

Valves manifold, SMC series SS3YJ5 ((Type 41) or equivalent

Tubing, SMC series TU (polyurethane) or equivalent

Tubing, SMC series T (nylon) or equivalent

PLC, Allen-Bradley SLC 5/04 modular processor or equivalent

Power supply, Allen-Bradley 1746 or equivalent

I/O digital modules, Allen-Bradley 1746 or equivalent

Chassis module, Allen-Bradley 1746 or equivalent

RSLogix 500, Software Rockwell Automation or equivalent

Vacuum pump, Vaccon J series or equivalent

Round disc target, 0.75 inch OD×0.062 inch thick (ESPI, Electronic Space Products International) or equivalent

Syringe holder—TEFLON, from DuPont

Addresses

AAPER Alcohol, P.O. Box 339 Shelbyville, Ky. 40066 USA

Air-Tite Products Co, Inc., 565 Central Drive, Virginia Beach, Va. 23454 USA

Alfa Aesar, 30 Bond Street, Ward Hill, Mass. 01835 USA

Allen-Bradley/Rockwell Automation, 1201 South Second Street, Milwaukee, Wis. 53204 USA

American Pharmaceutical Partners, 3 Parkway North Center, Deerfiel Ill. 60015 USA

ESPI, Electronic Space Products International, 1050 Benson Way, Ashland, Oreg. 97520 USA

Millipore Corporation, 290 Concord Road, Billerica, Mass. 01821 U.S.A.

Sigma Aldrich, PO Box 14508, St. Louis, Mo. 63178 USA

SMC Corporation/US Headquarters, 3011 North Franklin Road, Indianapolis Ind. 46226 USA

Vaccon Co Inc, 32 Rear Spring St, Medfield, Mass. 02052 USA

Waters Corp, 34 Maple Street, Milford, Mass. 01757 USA

Advantageously this discovery provides a new way to operate a copper recovery process which will enhance the recovery process, decrease the cost and optimize the efficiency.

Those of skill in the art will recognize that process conditions, reactions and operating setup will be apparent to one of skill in the art after reading this specification and claims and drawings such that the process will be setup and operated such to make it operable and to achieve its intended purpose. It is understood that software, hardware, valves, connectors and connections are fully communicative and operable and operationally enabled in accordance with this discovery. The purified isotopes are thus recovered in this discovery for further use.

Advantageously, this discovery provides a process with minimal or no human direct exposure to radioactivity and with minimal or no human physical intervention in the process.

While the discovery has been described in terms of various specific embodiments, those skilled in the art will recognize that the discovery can be practiced with modification within the spirit and scope of the claims.

What is claimed is:

1. An automated functional process for separating ⁶⁰Cu, ⁶¹Cu and ⁶⁴Cu from a starting material independently respectively and recovering ⁶⁰Cu, ⁶¹Cu and ⁶⁴Cu separately and independently as purified recovered product(s) therefrom wherein automation of automated process is accomplished by utilizing an electronic control system wherein the electronic control system is computer operated thereby producing and recovering each of ⁶⁰Cu or ⁶¹Cu or ⁶⁴Cu separately and independently as a purified product.

2. A method in accordance with claim 1 wherein a ladder logic program instructs at least one of a timer, counter, and motion controller following or accordingly to a specific sequence and the computer is a PLC.

3. A method in accordance with claim 2 wherein the program provides analog signals and analog outputs.

4. A method in accordance with claim 3 wherein analog signals and analog outputs are used to instruct a temperature sequence.

5. A method in accordance with claim 4 wherein analog signals and analog output is used to monitor activity through processing.

6. A method in accordance with claim 5 wherein the software program providing said signals and analog outputs is computer controlled as by using a programmed PLC.

7. A method in accordance with claim 6 wherein the software program is responsive to process dynamics.

8. A functional automated method for separating a radioactive starting target material comprising ⁶⁰Cu containing ⁶⁰Ni, or a radioactive ⁶¹Cu containing ⁶¹Ni, or a radioactive ⁶⁴Cu containing ⁶⁴Ni therein which comprises dissolving that irradiated ⁶⁰Cu, or ⁶¹Cu, or ⁶⁴Cu respective starting material mixture in a solvent acid to form an acidic solubilized composition, feeding/loading the acidic solubilized composition onto an ion exchange column and removing an eluent comprising ⁶⁰Ni, or ⁶¹Ni, or ⁶⁴Ni ions respectively and recovering each of ⁶⁰Cu, ⁶¹Cu and ⁶⁴Cu as a separate and independent purified product respectively.

9. A method in accordance with claim 8 wherein automation of the method is accomplished by utilizing an electronic control system and the method uses a chromatographic system.

10. A method in accordance with claim 9 wherein a relay ladder logic program instructs at lease on timer, counter, and motion controllers according to a specific sequence.

11. A method in accordance with claim 10 wherein the program provides analog signals and analog outputs.

12. A method in accordance with claim 11 wherein analog signals and analog outputs are used to instruct the temperature sequence and to monitor activity throughout processing.

13. A method in accordance with claim 12 wherein the software program providing the signals and analog outputs is computer controlled.

14. An automated separation system comprising a programmed PLC comprising a chromatographic separation zone further comprising a resin having a sufficient distinctive resin binding capacity for a ⁶⁰Cu, or ⁶¹Cu, or ⁶⁴Cu over ⁶⁰Ni, or ⁶¹Ni, or ⁶⁴Ni respectively and having a separation capability effective to substantially chromatographically separate precursor ⁶⁰Ni, or ⁶¹Ni, or ⁶⁴Ni from ⁶⁰Cu, or ⁶¹Cu, or ⁶⁴Cu respectively.

15. A method in accordance with claim 14 wherein automation of the automated method utilizes an electronic control system and the system is chromatographic and ⁶⁰Cu, ⁶¹Cu and ⁶⁴Cu are recovered as purified product(s) therefrom

16. A method in accordance with claim 15 wherein a ladder program instructs timers, counters, and motion controllers according to a pre-determined sequence.

17. A method in accordance with claim 16 wherein the program provides specific analog signals and analog outputs.

18. A method in accordance with claim 17 wherein analog signals and analog outputs are used to instruct the temperature sequence and to monitor activity throughout processing.

19. A method in accordance with claim 18 wherein the software program providing said signals and analog outputs is computer controlled.

20. An automated process for synthetically forming a ⁶⁰Cu, or ⁶¹Cu, or ⁶⁴Cu labeled product comprising loading purified ⁶⁰Cu, or ⁶¹Cu, or ⁶⁴Cu in 0.5N HCl solution onto a concentrating assembly, removing an about 0.5N HCl eluent, adding 3N HCl thereto, and admixing about 10-μL of ligand solution with the highly purified ⁶⁰Cu, or ⁶¹Cu, or ⁶⁴Cu in the concentrating assembly and recovering each of ⁶⁰Cu, ⁶¹Cu and ⁶⁴Cu as an individual purified recovery products as a result of the process.

21. A method in accordance with claim 20 wherein the mixture formed with Cu-60, or Cu-61, or Cu-64 in about 3N HCl/ligand is loaded onto a purifying cartridge removing an about 3N HCl eluent.

22. A method in accordance with claim 21 wherein a further purification step comprises loading 10-mL sterile water into the reaction assembly to remove the ⁶⁰Cu, or ⁶¹Cu, or ⁶⁴Cu labeled product which is adherent in the reaction assembly.

23. A method in accordance with claim 22 wherein ethanol is loaded onto the purifying cartridge.

24. A method in accordance with claim 23 wherein the assembly comprises concentrating and purifying cartridges.

25. A method in accordance with claim 24 wherein the system comprises a line or reaction chamber comprising a lumen for suitably reacting products therein.

26. A method in accordance with claim 25 wherein automation is accomplished by utilizing an electronic control system.

27. A method in accordance with claim 26 wherein the assembly has a processing unit and PLC which are enclosed within a 19″W×12″D×25″H enclosure and the enclosure placed within a hot cell.

28. A method in accordance with claim 27 wherein a relay ladder logic program instructs timers, counters, and motion controllers according to a specific sequence.

29. A method in accordance with claim 28 wherein analog signals and analog outputs are used to instruct the temperature sequence and to monitor activity throughout processing.

30. A method in accordance with claim 29 wherein the software program providing said signals and analog outputs is loaded into a PLC which controls the process.

31. A method of controlling an automated process for synthetically forming a ⁶⁰Cu, or ⁶¹Cu, or ⁶⁴Cu labeled product comprises loading purified ⁶⁰Cu, or ⁶¹Cu, or ⁶⁴Cu in 0.5N HCl solution onto a concentrating assembly, removing an about 0.5N HCl eluent, adding 3N HCl thereto, and admixing about 10-μL of ligand solution with the highly purified ⁶⁰Cu, or ⁶¹ Cu, or ⁶⁴Cu in the concentrating assembly forming a reaction system which comprises forming a database containing sequence control information and using that database to control the process.

32. A method in accordance with claim 31 wherein the process is a chromatographic column and ⁶⁰Cu, ⁶¹Cu and ⁶⁴Cu are recovered as purified product(s),

33. A method in accordance with claim 32 wherein the column is a separation column for copper isotopes.

34. A database comprising sequence process valve instruction for controlling an automated process for synthetically forming a ⁶⁰Cu, or ⁶¹Cu, or ⁶⁴Cu labeled product which comprises loading purified ⁶⁰Cu, or ⁶¹Cu, or ⁶⁴Cu in 0.5N HCl solution onto a concentrating assembly, removing an about 0.5N HCl eluent, adding 3N HCl thereto, and admixing about 10-μL of ligand solution with the highly purified ⁶⁰Cu, or ⁶¹Cu, or ⁶⁴Cu in the concentrating assembly forming a reaction system and controlling the process.

35. A method in accordance with claim 34 which comprises constructing a database for use in controlling an automated process for forming a copper labeled product which comprises loading value sequence instructions into a database.

36. A method of controlling an automated process for preparing a copper nuclide by utilizing the database of claim 35.

37. A method in accordance with claim 36 wherein the database comprises a sequence of valve openings and valve closings.

38. A method in accordance with claim 2 wherein systems of the program are digital.
